(19) United States
(12) Patent Application Publication
(10) Pub. No.: US 2003/0055297 A1
(43) Pub. Date: Mar. 20, 2003

(54) Process for the preparation of substituted pyrrolidine neuraminidase inhibitors

(76) Inventors: Steven J. Wittenberger, Mundelein, IL (US); Lakshmi Bhagavatula, Vernon Hills, IL (US); David A. DeGoey, Kenosha, WI (US); John DeMattei, Longmont, CO (US); Ashok K. Gupta, Gurnee, IL (US); David R. Hill, Gurnee, IL (US); Sukumar Manna, Libertyville, IL (US); Maureen Ann McLaughlin, Gurnee, IL (US); Paul J. Nichols, Longmont, CO (US); Premchandran H. Ramiya, Naperville, IL (US); Michael W. Rasmussen, Kenosha, WI (US); Zhenping Tian, Grayslake, IL (US)

Correspondence Address: STEVEN F. WEINSTOCK; ABBOTT LABORATORIES, 100 ABBOTT PARK ROAD, DEPT. 377/AP6A, ABBOTT PARK, IL, 60064-6008, US

(21) Appl. No.: 10/115,310

(22) Filed: Apr. 3, 2002

Related U.S. Application Data
(60) Provisional application No. 60/281,171, filed on Apr. 3, 2001.

(51) Int. Cl. C07C 29/48

(52) U.S. Cl. 568/815; 568/867

(57) ABSTRACT

A process for the preparation of neuraminidase inhibitors having structural formula (28)

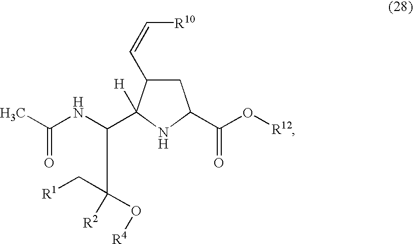

or therapeutically acceptable salts thereof, in which R¹ is alkyl, cycloalkyl, cycloalkylalkyl, or arylalkyl; R² is alkyl, cycloalkyl, cycloalkylalkyl, or arylalkyl; R⁴ is alkyl, cycloalkyalkyl, or aryl-(C₂-C₄-alkyl); R¹⁰ is methyl, ethyl, iso-propyl, or vinyl; and

R¹² is hydrogen or alkyl and intermediates useful for the process are disclosed.

This application claims priority to co-pending U.S. Provisional Application Serial No. 60/281,171, filed Apr. 3, 2001, the specification of which is hereby incorporated by reference into this application.

### TECHNICAL FIELD

This invention is directed to a process for making substituted pyrrolidine neuraminidase inhibitors and to intermediates which are useful in the process.

### BACKGROUND OF THE INVENTION

Drugs such as zanamivr (Relenza®) are known in the art as agents which inhibit neuraminidases and are therefore potentially useful for the treatment and prophylaxis of influenza (N Engl J Med 2000; 343(18): 1282-9).

A novel series of substituted pyrrolidine inhibitors of neuraminidases is also disclosed in commonly owned, co-pending U.S. patent application Ser. No. 09/421,787, a subset series of which is illustrated by a compound having structural formula (28)

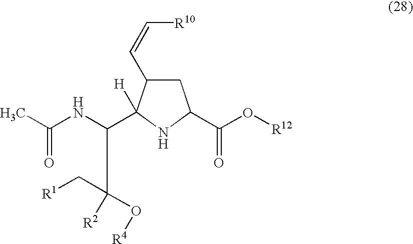

in which R¹ is alkyl, cycloalkyl, cycloalkylalkyl, or arylalkyl; R² is alkyl, cycloalkyl, cycloalkylalkyl, or arylalkyl; R⁴ is alkyl, cycloalkyalkyl, or aryl-(C₂-C₄-alkyl); R¹⁰ is methyl, ethyl, iso-propyl, or vinyl; and R¹² is hydrogen or alkyl.

A more cost-effective preparation, however, is needed for the manufacture of the compounds of this subset series in the quantities required for clinical development.

### DISCLOSURE OF THE INVENTION

This invention, therefore, is directed to a synthesis of inhibitors of neuraminidases in which column chromatographic purification is omitted; in which steps are conducted sequentially, in situ, and continuously; and in which critical reaction parameters such as agents employed and ratios of the same, solvents, temperatures, and times are optimized to provide a more cost-efficient synthesis than that disclosed in the '787 application.

The term “steps conducted sequentially” means conduction of a plurality of steps without work-up between the steps.

The term “steps conducted in situ” means conduction of a plurality of steps with a workup between the steps and during which the product remains in solution. Steps conducted in situ can be accompanied by solvent exchange, during which one solvent or solvents is substantially exchanged for another solvent or solvents and during which exchange the product remains in solution.

The term “steps conducted continuously” means the conduction of a step followed by work-up and solvent removal before beginning a subsequent step.

It is meant to be understood that in the hierarchy of preferentially for the practice of this invention, methods comprising steps which are conducted sequentially are preferred over methods comprising steps conducted in situ, which are, in turn, preferred over methods comprising steps conducted continuously.

It is also meant to be understood also that the terms L¹, R¹-R¹⁵, P¹, Q¹, X¹, each meant to represent groups of radicals, have the following meanings: “alkoxy” means an alkyl radical attached to the parent molecular group through an oxygen atom; “alkoxycarbonyl” means an alkoxy radical attached to the parent molecular group through a carbonyl; “alkyl” means a saturated, monovalent straight or branched hydrocarbon radical having one to six carbon atoms; “aryl” means a six-membered aromatic, carbocyclic ring which is unsubstituted or substituted by independent replacement of a hydrogen atom or atoms thereon by an alkyl, alkoxy, halo, or nitro radical; “arylalkyl” means an alkyl radical derivatized by the replacement of a hydrogen atom thereon by an aryl radical; “aryl-(C₂-C₄-alkyl)” means an alkyl radical having two, three, or four carbon atoms derivatized by the replacement of a hydrogen atom thereon by an aryl radical; “carbonyl” means —C(═O)—; “cycloalkyl” means a saturated, cyclic hydrocarbon radical having three to six carbon atoms; “cycloalkylalkyl” means an alkyl radical derivatized by replacement of a hydrogen atom thereon by a cycloalkyl radical; “halo” means a fluoride (F), chloride (Cl), or bromide (Br) radical; “haloalkyl” means an alkyl radical derivatized by independent replacement of one to six hydrogen atoms thereon by a halo radical; “hydroxy” means —OH; “nitro” means —NO₂; and “vinyl” means —C(H)═C(H)₂.

In a first embodiment of this invention, then, is disclosed a process for making a compound having structural formula (5)

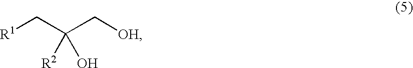

in which R¹ is alkyl, cycloalkyl, cycloalkylalkyl, or arylalkyl; and

R² is alkyl, cycloalkyl, cycloalkylalkyl, or arylalkyl, comprising the steps of:

(a) reacting a compound having structural formula (1)

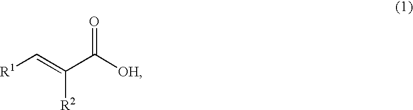

a preferred embodiment of which is substantially pure (2E)-2-methyl-2-pentenoic acid,

a first acid, a first esterifying agent, and, optionally, a trialkylorthoformate to provide a compound having structural formula (2)

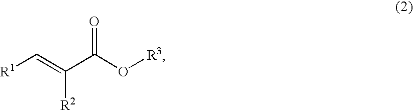

in which R³ is C₁-C₄ alkyl,

a preferred embodiment of which is substantially pure methyl (2E)-2-methyl-2-pentenoate;

(b) reacting the product of step (a) and a first reducing agent to provide a compound having structural formula (3)

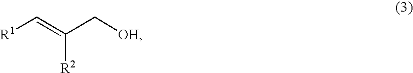

and a preferred embodiment of which is substantially pure (2E)-2-methyl-2-penten-1-ol;

(c) reacting the product of step (b) and a titanium iso-propoxidedialkyl tartrate complex;

(d) reacting the product of step (c) and tert-butyl hydroperoxide to provide a compound having structural formula (4)

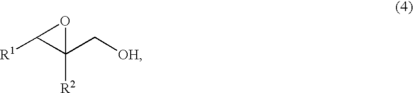

a preferred embodiment of which is a compound having the stereochemistry illustrated by a compound having structural formula (4-a)

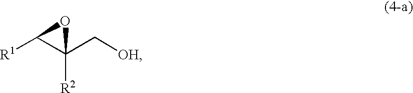

and a more preferred embodiment of which is substantially pure, enantiomerically enriched 3,4-anhydro-1,2-dideoxy-4-methyl-D-threo-pentitol;

and

(e) reacting the product of step (d) and a second reducing agent to provide the compound having structural formula (5),

a preferred embodiment of which is the compound having the stereochemistry illustrated by a compound having structural formula (5-a)

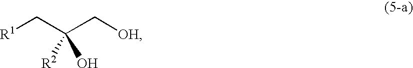

and a more preferred embodiment of which is substantially pure, enantiomerically enriched (2S)-2-methyl-1,2-pentanediol;

and

(f) optionally isolating the compound having structural formula (5).

In a preferred first embodiment, steps (c) and (d) are conducted sequentially, and steps (d) and (e) are conducted continuously.

Examples of first acids useful for the practice of step (a) include hydrochloric acid, perchloric acid, phosphoric acid, sulfuric acid, trifluoroacetic acid, and the like.

A preferred acid for the practice of step (a) is sulfuric acid.

Examples of first esterifying agents useful for the practice of step (a) include iso-butylene, a C₁-C₄ alcohol such as methanol, ethanol, iso-propanol, tert-butyoxycarbonyl-2-(1-acetamido-2-methoxy-2-methyl)pentyl-3-(cis-propeny-1-yl)pyrrolidine-5-carboxylic acid tert-butyl ester, and the like.

A preferred first esterifying agent for the practice of step (a) is methanol.

Examples of trialkylorthoformates useful for the practice of step (a) include trimethylorthoformate, triethylorthoformate, and tri-iso-propylorthoformate.

A preferred trialkylorthoformate for the practice of step (a) is trimethylorthoformate.

Step (a) is conducted at a temperature between about 25° C. and 100° C. in a C₁-C₅ alcohol solvent such as methanol, ethanol, iso-propanol, n-butanol, and the like.

In a preferred embodiment, step (a) is conducted at about 60° C. in essentially methanol.

Examples of first reducing agents useful for the practice of step (b) include lithium borohydride, lithium borohydride.pyrrolidine, lithium triethylborohydride, lithium triethylborohydride, potassium triethylborohydride, potassium tri(sec-butyl)borohydride, potassium triphenylborohydride, aluminum hydride, aluminum hydride.triethylamine, di(iso-butyl)aluminum hydride, iso-propoxyaluminum hydride, tert-butoxyaluminum hydride, di(tert-butoxy)aluminum hydride, lithium aluminum hydride, lithium aluminum hydride.N-methylpyrrolidine, sodium aluminum hydride, sodium diethylaluminum hydride, bis(2-methoxyethoxy)aluminum hydride, lithium di(iso-butyl)aluminum hydride, and the like.

A preferred first reducing agentfor the practice of step (b) is di(iso-butyl)aluminum hydride.

Step (b) is conducted at a temperature between about −60° C. and about −50° C. in a solvent such as diethyl ether, hexane, heptane, tetrahydrofuran, or a mixture thereof.

In a preferred embodiment, step (b) is conducted at about −60° C. in essentially hexane.

In another part of the first embodiment of this invention, the titanium iso-propoxide.dialkyl tartrate used for the practice of step (c) is prepared by reacting titanium iso-propoxide and a dialkyl tartrate.

Preferably, the dialkyl tartrate employed for the practice of step (c) is D-dimethyl tartrate, D-diethyl tartrate, or D-diisopropyl tartrate.

More preferably the dialkyl tartrate employed for the practice of step (c) is D-dimethyl tartrate.

Step (c) is conducted at a temperature between about −10° C. and about −30° C. in a solvent such as dichloromethane, chloroform, carbon tetrachloride, or a mixture thereof.

In a preferred embodiment, step (c) is conducted at about −20° C. in essentially dichloromethane.

Step (d) is conducted at a temperature of between about −30° C. and about −50° C. in a solvent such as carbon tetrachloride, chloroform, decane, dichloromethane, hexane, heptane, pentane, or a mixture thereof.

In a preferred embodiment, step (d) is conducted at about −40° C. in essentially a mixture of decane and dichloromethane.

In another part of the first embodiment of this invention, both the titanium iso-propoxidedialkyl tartrate complex and the tert-butyl hydroperoxide in steps (c) and (d), respectively, are each present in about 0.3 molar equivalents per molar equivalent of the compound having structural formula (3), and the compound having formula (4) has an enantiomeric excess of between about 86% and 89%. In a preferred embodiment of these steps, both the titanium iso-propoxidedialkyl tartrate complex and the tert-butyl hydroperoxide in steps (c) and (d), respectively, are each present in about 1.0 molar equivalent per molar equivalent of the compound having structural formula (3), and the compound having formula (4) has an enantiomeric excess of between about 94% and about 97%.

Examples of second reducing agents useful for the practice of step (e) include borane.dimethylsulfide, borane.tetrahydrofuran, borane and boron trifluoride.diethyletherate, borane.sodium borohydride, lithium borohydride, lithium borohydride.pyrrolidine, lithium triethylborohydride, lithium triethylborohydride, potassium triethylborohydride, potassium tri(sec-butyl)borohydride, potassium triphenylborohydride, aluminum hydride, aluminum hydride.triethylamine, di(iso-butyl)aluminum hydride, iso-propoxyaluminum hydride, tert-butoxyaluminum hydride, di(tert-butoxy)aluminum hydride, lithium aluminum hydride, lithium aluminum hydride.N-methylpyrrolidine, sodium aluminum hydride, sodium diethylaluminum hydride, bis(2-methoxyethoxy)aluminum hydride, lithium di(iso-butyl)(butyl)aluminum hydride, and the like.

A preferred second reducing agent for the practice of step (e) is lithium aluminum hydride.

Step (e) is conducted at a temperature between about −30° C. and about −50° C. in a solvent such as diethylether, dioxane, tetrahydrofuran, or a mixture thereof.

In a preferred embodiment, step (e) is conducted at about −40° C. in essentially tetrahydrofuran.

In another preferred first embodiment, step (f) is conducted, and the product of step (e) is isolated by distillation.

A compound having structural formula (5) is useful as an intermediate for making a compound having structural formula (12)

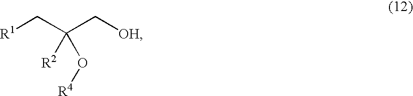

in which R¹ is alkyl, cycloalkyl, cycloalkylalkyl, or arylalkyl;

R² is alkyl, cycloalkyl, cycloalkylalkyl, or arylalkyl; and

R⁴ is alkyl, cycloalkyalkyl, or aryl-(C₂-C₄-alkyl).

In a second embodiment of this invention, therefore, is disclosed a process for making a compound having structural formula (12) from the compound having structural formula (5) comprising, in toto, the steps of:

(g) reacting the compound having structural formula (5)

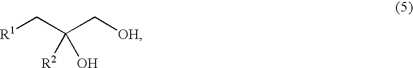

a preferred embodiment of which is a compound having the stereochemistry illustrated by a compound having structural formula (5-a)

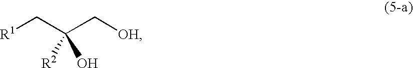

and a more preferred embodiment of which is substantially pure, enantiomerically enriched (2S)-2-methyl-1 ,2-pentanediol,

a first base, and a selectively removable hydroxy protecting group precursor to provide a compound having structural formula (6)

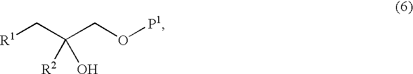

in which P¹ is a selectively removable hydroxy protecting group,

a preferred embodiment of which is a compound having the stereochemistry illustrated by a compound having structural formula (6-a)

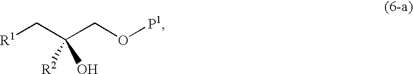

and a more preferred embodiment of which is substantially pure, enantiomerically enriched (2S)-1-(benzyloxy)-2-methyl-2-pentanol;

(h) reacting the product of step (g), an alkylating agent, and the first base to provide a compound having structural formula (7)

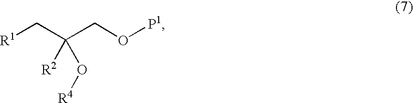

a preferred embodiment of which is a compound having the stereochemistry illustrated by a compound having structural formula (7-a)

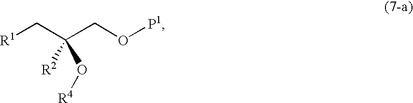

and a more preferred embodiment of which is substantially pure, enantiomerically enriched ((((2S)-2-methoxy-2-methylpentyl)oxy)methyl)benzene;

(i) reacting the product of step (h) and a hydroxy protecting group removal agent to provide the compound having structural formula (12),

a preferred embodiment of which is a compound having the stereochemistry illustrated by a compound having structural formula (12-a)

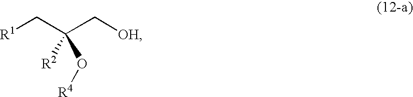

a more preferred embodiment of which is a compound having the stereochemistry illustrated by a compound having structural formula (12-b),

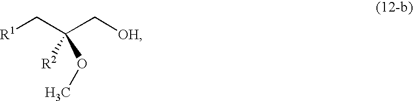

and a still more preferred embodiment of which is substantially pure, enantiomerically enriched (2S)-2-methoxy-2-methyl-1 -pentanol;

and

(j) optionally isolating the compound having structural formula (12).

In a preferred second embodiment, steps (g) and (h) are conducted sequentially, and steps (h) and (i) are conducted continuously.

Examples of first bases useful for the practice of step (g) and step (h) include dibutyltin oxide, lithium diisopropylamide, sodium hydride, potassium hydride, lithium bis(trimethylsilylamide), sodium bis(trimethylsilylamide), potassium bis(trimethylsilylamide), lithium tert-butoxide, sodium tert-butoxide, potassium tert-butoxide, lithium tert-amylate, sodium tert-amylate, potassium tert-amylate, and the like.

A preferred first base for the practice of step (g) and step (h) is sodium hydride.

Examples of selectively removable hydroxy protecting group precursors for the practice of step (g) include methoxymethyl bromide, methoxymethyl chloride, methylthiomethyl bromide, methylthiomethyl chloride, (phenyldimethylsilyl)methoxymethyl bromide, (phenyldimethylsilyl)methoxymethyl chloride, benzyloxymethyl bromide, benzyloxymethyl chloride, para-methoxybenzyloxymethyl bromide, para-methoxybenzyloxymethyl chloride, para-nitrobenzyloxymethyl bromide, para-nitrobenzyloxymethyl chloride, ortho-nitrobenzyloxymethyl bromide, ortho-nitrobenzyloxymethyl chloride, (4-methoxyphenoxy)methyl bromide, (4-methoxyphenoxy)methyl chloride, tert-butoxymethyl bromide, tert-butoxymethyl chloride, 4-pentenyloxymethyl bromide, 4-pentenyloxymethyl chloride, siloxymethyl bromide, siloxymethyl chloride, 2-methoxyethoxymethyl bromide, 2-methoxyethoxymethyl chloride, 2,2,2-trichloroethoxymethyl bromide, 2,2,2-trichloroethoxymethyl chloride, bis(2-chloroethoxy)methyl bromide, bis(2-chloroethoxy)methyl chloride, 2-(trimethylsilyl)ethoxymethyl bromide, 2-(trimethylsilyl)ethoxymethyl chloride, 1-ethoxyethyl bromide, 1-ethoxyethyl chloride, 1-(2-chloroethoxy)ethyl bromide, 1-(2-chloroethoxy)ethyl chloride, 1-(2-(trimethylsilyl)ethoxy)ethyl bromide, 1-(2-(trimethylsilyl)ethoxy)ethyl chloride, para-methoxybenzyl bromide, para-methoxybenzyl chloride, 1-methyl-1-methoxyethyl bromide, 1-methyl-1-methoxyethyl chloride, 1-methyl-1-benzyloxyethyl bromide, 1-methyl-1-benzyloxyethyl chloride, 1-methyl-1-benzyloxy-2-fluoroethyl bromide, 1-methyl-1-benzyloxy-2-fluoroethyl chloride, 1-methyl-1-phenoxyethyl bromide, 1-methyl-1-phenoxyethyl chloride, 2,2,2-trichloroethyl bromide, 2,2,2-trichloroethyl chloride, benzyltrichloroacetimidate, 2-trimethylsilylethyl bromide, 2-trimethylsilylethyl chloride, 2-(benzylthio)ethyl bromide, 2-(benzylthio)ethyl chloride, 2-(phenylselenyl)ethyl bromide, 2-(phenylselenyl)ethyl chloride, allyl bromide, allyl chloride, propargy bromide, propargy chloride, benzyl bromide, benzyl chloride, 3,4-dimethoxybenzyl bromide, 3,4-dimethoxybenzyl chloride, ortho-nitrobenzyl bromide, ortho-nitrobenzyl chloride, para-nitrobenzyl bromide, para-nitrobenzyl chloride, para-halobenzyl bromide, para-halobenzyl chloride, 2,6-dichlorobenzyl bromide, 2,6-dichlorobenzyl chloride, para-cyanobenzyl bromide, para-cyanobenzyl chloride, para-phenylbenzyl bromide, 2,6-difluorobenzyl bromide, para-acylaminobenzyl bromide, para-acylaminobenzyl chloride, para-azidobenzyl bromide, para-azidobenzyl chloride, 4-azido-3-chlorobenzyl bromide, 4-azido-3-chlorobenzyl chloride, 2-trifluoromethylbenzyl bromide, 2-trifluoromethylbenzyl chloride, para-(methylsulfinyl)benzyl bromide, para-(methylsulfinyl)benzyl chloride, 2-picolyl bromide, 2-picolyl chloride, 4-picolyl bromide, 4-picolyl chloride, 3-methyl-2-picolyl N-oxido bromide, 3-methyl-2-picolyl N-oxido chloride, 2-quinolinylmethyl bromide, 2-quinolinylmethyl chloride, 1-pyrenylmethyl bromide, 1-pyrenylmethyl chloride diphenylmethyl bromide, diphenylmethyl chloride, para, para′-dinitrobenzhydryl bromide, para,para′-dinitrobenzhydryl chloride, 5-dibenzosuberyl bromide, 5-dibenzosuberyl chloride, triphenylmethyl bromide, triphenylmethyl chloride, α-naphthyldiphenylmethyl bromide, α-naphthyldiphenylmethyl chloride, para-methoxyphenyldiphenylmethyl bromide, para-methoxyphenyldiphenylmethyl chloride, di(para-methoxyphenyl)phenylmethyl bromide, di(para-methoxyphenyl)phenylmethyl chloride, tri(para-methoxyphenyl)methyl bromide, tri(para-methoxyphenyl)methyl chloride, 4-(4′-bromophenacyloxy)phenyldiphenylmethyl bromide, 4-(4′-bromophenacyloxy)phenyldiphenylmethyl chloride, 4,4′,4″-tris(4,5-dichlorophthalimidophenyl)methyl bromide, 4,4′,4″-tris(4,5-dichlorophthalimidophenyl)-methyl chloride, 4,4′,4″-tris(levulinoyloxyphenyl)methyl bromide, 4,4′,4″-tris(levulinoyl-oxyphenylmethyl chloride, 4,4′,4″-tris(benzoyloxyphenyl)methyl bromide, 4,4′,4″-tris(benzoyloxyphenyl)methyl chloride, 4,4′-dimethoxy-3″-(N-(imidazolyl-methyl))trityl bromide, 4,4′-dimethoxy-3″-(N-(imidazolylmethyl))trityl chloride, 4,4′-dimethoxy-3″-(N-(imidazolylethyl)carbamoyl)trityl bromide, 4,4′-dimethoxy-3″-(N-(imidazolylethyl)carbamoyl)trityl chloride, 1,1-bis(4-methoxyphenyl)-1′-pyrenylmethyl bromide, 1,1-bis(4-methoxyphenyl)-1′-pyrenylmethyl chloride, 9-anthryl bromide, 9-anthryl chloride, 9-(9-phenyl)xanthenyl bromide, 9-(9-phenyl)xanthenyl chloride, 9-(9-phenyl-10-oxo)anthryl bromide, 9-(9-phenyl-10-oxo)anthryl chloride, 1,3-benzodithiolan-2-yl bromide, 1,3-benzodithiolan-2-yl chloride, benzisothiazolyl S,S-dioxido bromide, benzisothiazolyl S,S-dioxido chloride, trimethylsilyl bromide, trimethylsilyl chloride, triethylsilyl bromide, triethylsilyl chloride, triisopropylsilyl bromide, triisopropylsilyl chloride, dimethylisopropylsilyl bromide, dimethylisopropylsilyl chloride, diethylisopropylsilyl bromide, diethylisopropylsilyl chloride, dimethylthexylsilyl bromide, dimethylthexylsilyl chloride, tert-butyldimethylsilyl bromide, tert-butyldimethylsilyl chloride, tert-butyldiphenylsilyl bromide, tert-butyldiphenylsilyl chloride, tribenzylsilyl bromide, tribenzylsilyl chloride, tri-para-xylylsilyl bromide, tri-para-xylylsilyl chloride, triphenylsilyl bromide, triphenylsilyl chloride, diphenylmethylsilyl bromide, diphenylmethylsilyl chloride, di-tert-butylmethylsilyl bromide, di-tert-butylmethylsilyl chloride, tris(trimethylsilyl)silyl, tris(trimethylsilyl)silyl, (2-hydroxystyryl)dimethylsilyl bromide, (2-hydroxystyryl)dimethylsilyl chloride, (2-hydroxystyryl)diisopropylsilyl bromide, (2-hydroxystyryl)diisopropylsilyl chloride, tert-butylmethoxyphenylsilyl bromide, tert-butylmethoxyphenylsilyl chloride, tert-butoxydiphenylsilyl bromide, tert-butoxydiphenylsilyl chloride, and the like.

Preferred selectively removable hydroxy protecting group precursors for the practice of step (g) include benzyl chloride, benzyl bromide, diphenylmethyl chloride, diphenylmethyl bromide, para-methoxybenzyl chloride, para-methoxybenzyl bromide, 3,4-dimethoxybenzyl chloride, 3,4-dimethoxybenzyl bromide, ortho-nitrobenzyl chloride, ortho-nitrobenzyl bromide, para-nitrobenzyl chloride, para-nitrobenzyl bromide, 2,6-dichlorobenzyl chloride, 2,6-dichlorobenzyl bromide, 2,6-difluorobenzyl chloride, 2,6-difluorobenzyl bromide, 2-trifluoromethylbenzyl chloride, 2-trifluoromethylbenzyl bromide, para-cyanobenzyl chloride, para-cyanobenzyl bromide, para-acylaminobenzyl chloride, para-acylaminobenzyl bromide, para-phenylbenzyl chloride, para-phenylbenzyl bromide, para-azidobenzyl chloride, and para-azidobenzyl bromide, and benzyltrichloroacetimidate.

More preferred selectively removable hydroxy protecting group precursors for the practice of step (g) are benzyl chloride or benzyl bromide.

Examples of alkylating agents useful for the practice of step (h) are alkyl halides such as methyl chloride, methyl bromide, methyl iodide, ethyl chloride, ethyl bromide, ethyl iodide, iso-propyl chloride, iso-propyl bromide, iso-propyl iodide, and the like; alkyl trifluoromethanesulfonates such as methyl trifluoromethanesulfonate, ethyl trifluoromethanesulfonate, iso-propyl trifluoromethanesulfonate, and the like; dialkylsulfates such as dimethylsulfate, diethylsulfate, diisopropylsulfate, and the like; cycloalkyalkyl halides such as (chloromethyl)cyclopropane, (bromoethyl)cyclobutane, (iodopropyl)cyclopentane, and the like; (trifluoromethanesulfonylmethyl)cyclopropane, (trifluoromethanesulfonylethyl)cyclobutane, (trifluoromethanesulfonylpropyl)-cyclopentane, and the like; dicycloalkyalkyl sulfates such as di(cyclopropylmethyl)sulfate, di(cyclobutylethyl)sulfate, di(cyclopentylpropyl)sulfate, and the like; aryl-(C₂-C₄-alkyl) halides such as phenethylbromide, phenpropyl iodide, and the like; aryl-(C₂-C₄-alkyl) triflates such as trifluoromethyl 2-phenylethoxysulfone, trifluoromethyl 2-phenylpropoxysulfone, and the like; and di-(aryl-(C₂-C₄-alkyl)) sulfates such as di(phenethyl)sulfate, and the like.

A preferred alkylating agent for the practice of step (h) is methyl iodide.

Steps (g) and (h) are conducted at a temperature between about −10° C. and about 30° C. in a solvent such as diethyl ether, tetrahydrofuran, dioxane, 1,2-dimethoxyethane, N,N-dimethylformamide, toluene, dimethylsulfoxide, acetonitrile, or a mixture thereof.

In a preferred embodiment, steps (g) and (h) are conducted at about 0° C. in tetrahydrofuran.

Examples of hydroxy protecting group removal agents useful for the practice of step (i) are hydrogen gas and a hydrogenolysis catalyst such as palladium metal, palladium metal on carbon, palladium metal on neutral alumina, palladium metal on acidic alumina, palladium metal on basic alumina, palladium(II) acetate, palladium(II) chloride, palladium(II) hydroxide on carbon, and the like.

A preferred hydrogenolysis catalyst for the practice of step (i) is palladium(II) hydroxide on carbon.

Step (i) is conducted at a temperature of between about 0° C. and about 30° C., at a pressure of about 10 pounds per square inch to about 80 pounds per square inch, in a solvent such as acetone, diethyl ether, dioxane, methanol, ethanol, iso-propanol, tert-butanol, ethyl acetate, iso-propyl acetate, methylethyl ketone, tetrahydrofuran, or a mixture thereof.

In a preferred embodiment, step (i) is conducted at about 25° C., at a pressure of about 40 pounds per square inch, in essentially tetrahydrofuran.

In another preferred second embodiment, step (j) is conducted, and the product of step (i) is isolated by distillation.

In a third embodiment of this invention is disclosed a process for making the compound having structural formula (12-b)

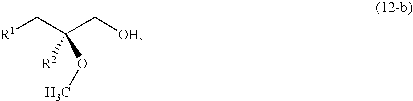

having the stereochemistry illustrated therein, in which

R¹ is alkyl, cycloalkyl, cycloalkylalkyl, or arylalkyl; and

R² is alkyl, cycloalkyl, cycloalkylalkyl, or arylalkyl, comprising the steps of:

(k) reacting a compound having structural formula (8)

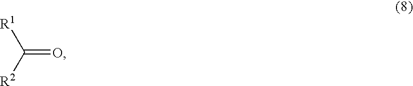

a preferred embodiment of which is 2-pentanone,

bromoform, potassium hydroxide, and methanol, at a temperature of between about 50° C. and about 70° C., to provide a compound having structural formula (9)

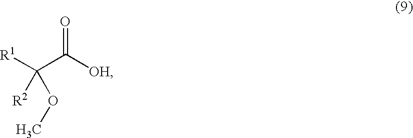

a preferred embodiment of which is (±)-2-methoxy-2-methylpentanoic acid,

(l) reacting the product of step ), a second esterifying agent and, optionally, an esterification promotion agent to provide a compound having structural formula (10)

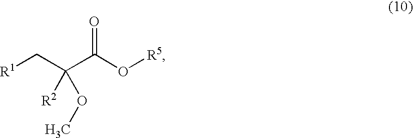

in which R⁵ is alkyl, C₂-C₄-haloalkyl, cycloalkylalkyl, or arylalkyl,

a preferred embodiment of which is substantially pure (±)-methyl 2-methoxy-2-methylpentanoate;

(m) reacting the compound having structural formula (10) and an esterase to provide either an enantiomerically enriched compound having the stereochemistry illustrated by a compound having structural formula (11-a)

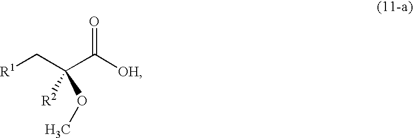

a preferred embodiment of which is substantially pure, enantiomerically enriched (2S)-2-methoxy-2-methylpentanoic acid, or an enantiomerically enriched compound having the stereochemistry illustrated by a compound having structural formula (11-b)

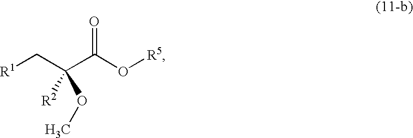

a preferred embodiment of which is substantially pure, enantiomerically enriched (2S)-methyl 2-methoxy-2-methylpentanoate;

(n) reacting either the compound having structural formula (11-a) or the compound having structural formula (11-b) and a third reducing agent to provide the compound having structural formula (12-b), a preferred embodiment of which is substantially pure, enantiomerically enriched (2S)-2-methoxy-2-methyl-1-pentanol;

and

(o) optionally isolating the compound having structural formula (12-b).

In a preferred third embodiment, steps (m) and (n) are conducted continuously, and step (o) is omitted.

In a preferred embodiment, step (k) is conducted at about 60° C.

Examples of second esterifying agents useful for the practice of step (l) include iso-butylene, a C₁-C₄ alcohol such as methanol, ethanol, and iso-propanol, tert-butyoxycarbonyl-2-(1-acetamido-2-methoxy-2-methyl)pentyl-3-(cis-propeny-1-yl)pyrrolidine-5-carboxylic acid tert-butyl ester, and the like, and, optionally an acid such as hydrochloric acid or sulfuric acid. Alternatively, the alcohol can be reacted with an esterification promotion agent such as methyl chloroformate, ethyl chloroformate, iso-butyl chloroformate, and the like, and a base such as N-methylmorpholine and the like, to provide a mixed anhydride intermediate which, when reacted with an alcohol such as methanol, ethanol, or trifluoroethanol, provides a compound having structural formula (10).

A preferred second esterifying agent for the practice of step (l) is methanol and sulfuric acid.

Examples of esterases useful for the practice of step (m) are Amano PS 30 Lipase, Amano AY Candida Cylindracea, Aspergillus niger lipase, Aspergillus oryzae protease, Alcaligenes species lipase, Aspergillus species protease, Bacillus species protease, Candida antartica “A” lipase, Candida antartica “B” lipase, Candida lipolytica lipase, Candida Rugosa esterase, Candida Rugosa lipase, ChiroCLEC-CR, ChiroCLEC-BL, ChiroCLEC-PC, ChiroCLEC-EC, alpha-crymotypsin, Humicola lanuginosa lipase, Mucor melhel lipase, Papain, PeptiCLEC-TR, Penicillin acylase, Pig Liver enzyme, Porcine Liver esterase, Porcine Pancreatic lipase, Pseudomonas cepacia lipase, Rhizopus Arrhizus Lipase Type XI, Rhizopus delmar lipase, and the like.

Preferred esterases for the practice of step (1) are Candida Rugosa lipase, Pig Liver enzyme, Amano PS 30 Lipas, Amano AY Candida Cylindracea, Porcine Pancreatic lipase, and Rhizopus Arrhizus Lipase Type XI.

A more preferred esterase for the practice of step (m) is Candida Rugosa Lipase.

In another embodiment for the practice of step (m), the esterase concentration is between about 20 mg per mL and about 40 mg per mL. In a preferred embodiment for the practice of this step, the esterase concentration is about 36 mg per mL.

In still another embodiment for the practice of step (m), the pH of the solution is between about 5.5 and about 8.5. In a more preferred embodiment for the practice of this step, the pH is about 8.

Step (m) is conducted at a temperature of between about 20° C. and about 50° C. in a solvent such as acetone, acetonitrile, a C₁-C₅ alcohol such as methanol, ethanol, and iso-propanol, dimethylsulfoxide, dioxane, heptane, hexane, methylethyl ketone, tetrahydrofuran, toluene, water, or a mixture thereof.

In a preferred embodiment, step (m) is conducted at a temperature of about 37° C. in essentially a mixture of water and acetone.

Examples of third reducing agents useful for the practice of step (n) are borane.dimethylsulfide, borane.THF, sodium borohydride and iodine, aluminum hydride, aluminum hydridetriethylamine, di(iso-butyl)aluminum hydride, iso-propoxyaluminum hydride, tert-butoxyaluminum hydride, di(tert-butoxy)aluminum hydride, lithium aluminum hydride, lithium aluminum hydride.N-methylpyrrolidine, sodium aluminum hydride, sodium diethylaluminum hydride, bis(2-methoxyethoxy)aluminum hydride, and lithium di(iso-butyl)(butyl)aluminum hydride.

The choice of third reducing agents for the practice of step (n) depends on the intermediate. An intermediate having structural formula (11-a) will be more efficiently reduced with a borane agent while an intermediate having formula (11-b) will be more efficiently reduced with an aluminum hydride agent.

In a preferred embodiment for the practice of step (m), the product of step (m) is a compound having formula (11-a), and the third reducing agent is borane.dimethylsulfide.

Step (n) is conducted at a temperature between about 0° C. and about 50° C. in a solvent such as acetone, acetonitrile, a C₁-C₅ alcohol such as methanol, ethanol, and iso-propanol, dichloromethane, dimethyl sulfide, dimethylsulfoxide, diethyl ether, dioxane, heptane, hexane, methylethyl ketone, tetrahydrofuran, toluene, water, or a mixture thereof.

In a preferred embodiment, step (n) is conducted in essentially a mixture of dichloromethane and dimethyl sulfide at a temperature of about 25° C.

The compound having structural formula (12) is useful as an intermediate for making a compound having structural formula (15)

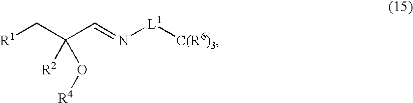

in which R¹ is alkyl, cycloalkyl, cycloalkylalkyl, or arylalkyl;

R² is alkyl, cycloalkyl, cycloalkylalkyl, or arylalkyl;

R⁴ is alkyl, cycloalkyalkyl, or aryl-(C₂-C₄-alkyl);

L¹ is N(R⁷), O, S, S(O), or SO₂;

R⁶ is aryl, furanyl, or, thienyl in which the aryl, the furanyl, and the thienyl are unsubstituted or substituted with one, two, or three substituents independently selected from the group consisting of alkyl, alkoxy, halo, and nitro; and

R⁷ is hydrogen, alkyl, aryl, or para-toluenesulfonyl.

In fourth embodiment of this invention, therefore, is disclosed a process for making a compound having structural formula (15) from the compound having structural formula (12) comprising, in toto, the steps of:

(p) reacting the compound having structural formula (12)

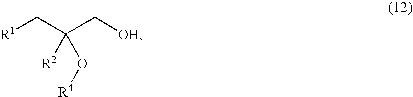

the preferred embodiment of which is the compound having the stereochemistry illustrated by the compound having structural formula (12-a)

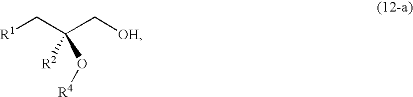

the more preferred embodiment of which is the compound having the stereochemistry illustrated by the compound having structural formula (12-b)

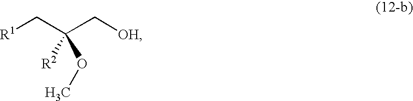

and the still more preferred embodiment of which is substantially pure, enantiomerically enriched (2S)-2-methoxy-2-methyl-1 -pentanol,

an oxidant, a second base, and, optionally, a first additive, in which the oxidant, the second base, and the additive are substantially soluble in the solvent or solvents in which this step is conducted, to provide a compound having structural formula (13)

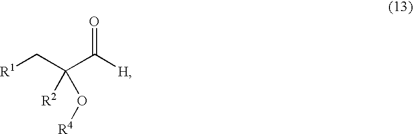

a preferred embodiment of which is a compound having the stereochemistry illustrated by a compound having structural formula (13-a)

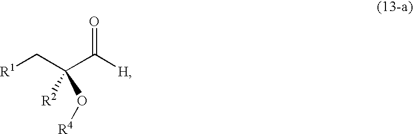

and a more preferred embodiment of which is substantially pure, enantiomerically enriched (2S)-2-methoxy-2-methylpentanal;

and

(q) reacting the product of step (p), a compound having structural formula (14)

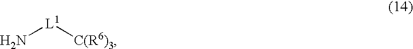

a preferred embodiment of which is substantially pure triphenylmethanesulfenamide,

a drying agent, and, optionally, a first acid catalyst to provide the compound having structural formula (15),

a preferred embodiment of which is a compound having the stereochemistry illustrated by a compound having structural formula (15-a)

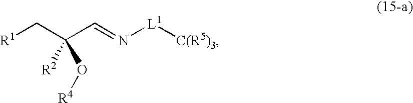

and a more preferred embodiment of which is substantially pure, enantiomerically enriched (1E,2S)-2-methoxy-2-methylpentanal S-tritylthioxime.

In a preferred fourth embodiment, steps (p) and (q) are conducted in situ.

Examples of oxidants useful for the practice of step (p) are an activated dimethyl sulfoxide agent such as dimethylsulfoxide and oxalyl chloride, dimethylsulfoxide.SO₃.pyridine, tetrapropylammonium perruthenate, 2,2,6,6-tetramethyl-1-piperidinyloxy, and the like.

Examples of first additives useful for the practice of step (p) are sodium hypochlorite/sodium bromide, potassium hypochlorite/potassium bromide, sodium hypochlorite/potassium bromide, potassium hypochlorite/sodium bromide, N-methylmorpholine-N-oxide, and the like.

In a preferred embodiment for the practice of step (p), the oxidant is an activated dimethyl sulfoxide agent, and the first additive is omitted. In a particularly preferred embodiment for the practice of this step, the oxidant is dimethylsulfoxide.SO₃.pyridine.

Examples of second bases useful for the practice of step (p) are diethylmethylamine, diisopropylethylamine, dimethylbutylamine, 1,4-dimethylpiperazine, 1,8-diazobicyclo[5.4.0]undec-7-ene, 1,5-diazobicyclo[4.3.0]non-5-ene, 1-methylpiperidine, pyrazine, pyridazine, pyrimidine, pyridine, tetramethylenediamino-methane, tributylamine, triethylamine, and tripropylamine.

In a preferred embodiment for the practice of step (p), the second base is triethylamine.

Step (p) is conducted at a temperature between about −10° C. and about 30° C. in a solvent such as chloroform, dichloromethane, diethyl ether, dioxane, ethyl acetate, dimethylsulfoxide, N,N-dimethylformamide, iso-propyl acetate, tetrahydrofuran, toluene, or a mixture thereof.

In a preferred embodiment, step (p) is conducted in essentially dichloromethane and dimethylsulfoxide at a temperature of about 0° C., after which the dimethylsulfoxide is substantially removed to provide the product of step (p) as a solution comprising between about 20% and about 40% by weight of the compound having structural formula (13) in dichloromethane. In a more preferred embodiment of this step, the product is a solution comprising about 30% by weight of the compound having structural formula (13) in dichloromethane.

Examples of drying agents useful for the practice of step (q) are 3 Å sieves, 4 Å sieves, anhydrous sodium sulfate, anhydrous magnesium sulfate, anhydrous potassium carbonate, and the like.

A preferred drying agent for the practice of step (q) is anhydrous sodium sulfate.

Examples of first acid catalysts for the practice of step (q) are camphorsulfonic acid, acetic acid/sodium acetate, pyridiniumpara-toluene sulfonate, and para-toluenesulfonic acid.

A preferred first acid catalyst for the practice of step (q) is pyridinium para-toluene sulfonate.

Step (q) is conducted at a temperature between about 0° C. and about 30° C. in a solvent such as chloroform, dichloromethane, diethyl ether, dimethylsulfoxide, dioxane, N,N-dimethylformamide, ethyl acetate, iso-propyl acetate, tetrahydrofuran, toluene, ortho-xylene, meta-xylene, para-xylene, tetrahydrofuran, or a mixture thereof.

In a preferred embodiment, step (q) is conducted at about 25° C. in essentially dichloromethane, after which the dichloromethane is substantially replaced with heptane to provide between about 40% and about 70% by weight of the compound having structural formula (15) in heptane. In a more preferred embodiment of this step, the product is a solution comprising essentially about 50% by weight of the compound having structural formula (15) in heptane.

In a fifth embodiment of this invention is disclosed a process for making substantially pure (±)-tert-butyl-4-hydroxy-2-oxo-pyrrolidinecarboxylate comprising, in toto, the steps of:

(r) reacting (±)-4-amino-3-hydroxy-n-butyric acid, a silating agent, and a third base to provide (±)-4-trimethylsilyloxy-2-oxo-pyrrolidine;

(s) reacting the (±)-4-trimethylsilyloxy-2-oxo-pyrrolidine and a tert-butylcarbonyloxy-introducing agent, the third base, and 4-dimethylaminopyridineto provide (±)-tert-butyl-4-trimethylsilyloxy-2-oxo-pyrrolidinecarboxylate;

(t) reacting the (±)-tert-butyl-4-trimethylsilyloxy-2-oxo-pyrrolidinecarboxylate and a desilylating agent to provide (±)-tert-butyl-4-hydroxy-2-oxo-pyrrolidinecarboxylate;

and

(u) isolating the substantially pure (±)-tert-butyl-4-hydroxy-2-oxo-pyrrolidine-carboxylate.

In a preferred fifth embodiment, steps (r), (s), (t), and (u) are conducted sequentially.

Examples of silating agents useful for the practice of step (r) are 1,1,1,3,3,3-hexamethyldisilazane, a compound of formula Si(R⁸)₃(Cl) in which each R⁸ is independently selected from the group consisting of alkyl and unsubstituted aryl, N,O-bis(trimethylsilyl)acetamide, and the like.

A preferred silating agent for the practice of step (r) is (±)-1,1,1,3,3,3-hexamethyldisilazane, which preferably is present between about 1 molar equivalent and about 2 molar equivalents per molar equivalent of the (±)-4-amino-3-hydroxy-n-butyric acid. In a more preferred embodiment of this step, the 1,1,1,3,3,3-hexamethyldisilazane is present in about 1.61 molar equivalents per molar equivalent of the (±)-4-amino-3-hydroxy-n-butyric acid.

Examples of third bases useful or the practice of step (r) are diethylenediamine, diethylmethylamine, diisopropylethylamine, dimethylbutylamine, 1,4-dimethylpiperazine, 1,8-diazobicyclo[5.4.0]undec-7-ene, 1,5-diazobicyclo[4.3.0]non-5-ene, 1-methylpiperidine, pyrazine, pyridazine, pyrimidine, pyridine, tetramethylenediamino-methane, tributylamine, triethylamine, and tripropylarnine.

A preferred third base for the practice of step (r) is pyridine, which preferably is present between about 0.01 to about 10 molar equivalents per molar equivalent of the (±)-4-amino-3-hydroxy-n-butyric acid. In a more preferred embodiment of this step, the pyridine is present in about 7.4 molar equivalents per molar equivalent of the (±)-4-amino-3-hydroxy-n-butyric acid.

Step (r) is conducted at a temperature between about 100° C. and about 150° C. in a solvent such as toluene, ortho-xylene, meta-xylene, para-xylene, or a mixture thereof.

In a preferred embodiment, step (r) is conducted at about 130° C. to about 140° C. in essentially a mixture comprising ortho-xylene, meta-xylene, and para-xylene, after which the solvent is substantially removed.

Examples of tert-butylcarbonyloxy-introducing agents useful for the practice of step (s) are di-tert-butyl dicarbonate, 1-(tert-butoxycarbonyl)-2-tert-butyl-3-methyl-4-imidazolidinone, (2-tert-butoxycarbonyloxyimino)-2-phenylacetonitrile), and the like.

A preferred tert-butylcarbonyloxy-introducing agent for the practice of step (s) is di-tert-butyl dicarbonate, which preferably is present in about 0.9 to about 1.5 molar equivalents per molar equivalent of the (±)-tert-butyl-4-hydroxy-2-oxo-pyrrolidinecarboxylate. In a more preferred embodiment of this step, the di-tert-butyl dicarbonate is present in about 1.2 molar equivalents per molar equivalent of the (±)-tert-butyl-4-hydroxy-2-oxo-pyrrolidinecarboxylate.

In another preferred embodiment, the 4-dimethylaminopyridine in step (s) is present in about 0.01 to about 0.1 molar equivalents per molar equivalent of the (±)-tert-butyl-4-hydroxy-2-oxo-pyrrolidinecarboxylate. In a more preferred embodiment of this step, the 4-dimethylaminopyridine is present in about 0.05 molar equivalents per molar equivalent of the (±)-tert-butyl-4-hydroxy-2-oxo-pyrrolidinecarboxylate.

Step (s) is conducted at a temperature between about −10° C. and about 30° C. in a solvent such as dioxane, ethyl acetate, iso-propyl acetate, tetrahydrofuran, or a mixture thereof.

In a preferred embodiment, step (s) is conducted at about 0° C. to about 25° C. in tetrahydrofuran, iso-propyl acetate, or a mixture thereof.

In a more preferred embodiment, step (s) is conducted at about 0° C. to about 25° C. in tetrahydrofuran.

In another more preferred embodiment, step (s) is conducted at about 0° C. to about 25° C. in iso-propyl acetate.

Examples of desilylating agents useful for the practice of step (t) are 2,3-dichloro-5,6-dicyano-1,4-benzoquinone in ethyl acetate and water, polymer-bound ammonium fluoride, tetrabutylammonium fluoride, triethylamine.trihydrofluoride, pyridine.HF, potassium carbonate in anhydrous methanol, and the like.

A preferred desilylating agent for the practice of step (t) is triethylainine.trihydrofluoride.

Step (t) is conducted at a temperature between about −10° C. and about 30° C. in a solvent such as dioxane, ethyl acetate, iso-propyl acetate, tetrahydrofuran, or a mixture thereof.

In a preferred embodiment, step (t) is conducted at about 0° C. to about 25° C. in tetrahydrofuran, iso-propyl acetate, or a mixture thereof.

In a more preferred embodiment, step (t) is conducted at about 0° C. to about 25° C. in tetrahydrofuran.

In another more preferred embodiment, step (t) is conducted at about 0° C. to about 25° C. in iso-propyl acetate.

In another preferred fifth embodiment, the practice of step (u) comprises isolation of the substantially pure (±)-tert-butyl-4-hydroxy-2-oxo-pyrrolidinecarboxylate by precipitation of the same from either essentially tetrahydrofuran or essentially iso-propyl acetate.

In still another preferred fifth embodiment, the practice of step (u) further comprises isolation of additional substantially pure (±)-tert-butyl-4-hydroxy-2-oxo-pyrrolidinecarboxylate by treatment of the tetrahydrofuran or the iso-propyl acetate with heptane and cooling the resulting solution to a temperature of about 0° C. to precipitate the additional substantially pure (±)-tert-butyl-4-hydroxy-2-oxo-pyrrolidine.

The compound having structural formula (15-a) and the (±)-tert-butyl-4-hydroxy-2-oxo-pyrrolidinecarboxylate are useful intermediates for making a compound having structural formula (19-a)

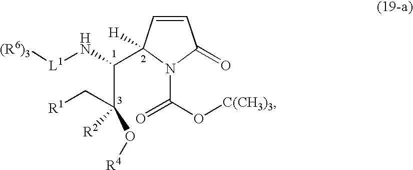

in which the absolute stereochemistry is illustrated therein, and in which R¹ is alkyl, cycloalkyl, cycloalkylalkyl, or arylalkyl;

R² is alkyl, cycloalkyl, cycloalkylalkyl, or arylalkyl;

R⁴ is alkyl, cycloalkyalkyl, or aryl-(C₂-C₄-alkyl);

L¹ is N(R⁷), O, S, S(O), or SO₂;

R⁶ is aryl, furanyl, or thienyl, in which the aryl, the furanyl, and the thienyl are unsubstituted or substituted with one, two, or three substituents independently selected from the group consisting of alkyl, alkoxy, halo, and nitro; and

R⁷ is hydrogen, alkyl, aryl, or para-toluenesulfonyl.

In a sixth embodiment of this invention, therefore, is disclosed a process for making a compound having structural formula (19-a) from the compound having structural formula (15-a) and the (±)-tert-butyl-4-hydroxy-2-oxo-pyrrolidine-carboxylate comprising, in toto, the steps of:

(v) reacting the (±)-tert-butyl-4-hydroxy-2-oxo-pyrrolidinecarboxylate, a fourth base, and a compound having structural formula (16)

X¹—SO₂—R⁹ (16),

in which R⁹ is alkyl, haloalkyl, and aryl, in which the aryl is unsubstituted or substituted with one, two, or three substituents independently selected from the group consisting of alkyl, alkoxy, halo, and nitro; and

X¹ is Br, Cl, or OSO₂—R⁹,

a preferred embodiment of which is methanesulfonyl chloride, to provide tert-butyl 2-oxo-3-pyrroline carboxylate;

(w) reacting the tert-butyl 2-oxo-3-pyrroline carboxylate, a fifth base, and a compound having structural formula (17)

(R⁸)₃SiOSO₂CF₃ (17),

in which each R⁸ is independently selected from the group consisting of alkyl and unsubstituted aryl,

a preferred embodiment of which is tert-butyldimethylsilyl trifluoromethanesulfonate,

to provide a compound having structural formula (18)

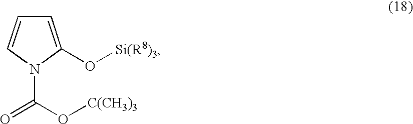

a preferred embodiment of which is tert-butyl 2-((tert-butyl(dimethyl)silyl)oxy)-1H-pyrrole-1-carboxylate;

(x) reacting the product of step (w), a second acid, and a substantially enantiomerically enriched compound having the structural formula (15-a)

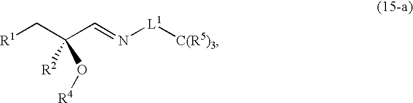

a preferred embodiment of which is substantially pure, substantially enantiomerically enriched (1E,2S)-2-methoxy-2-methylpentanal S-tritylthioxime,

to provide a mixture comprising two diastereomeric forms of a compound having formula (19)

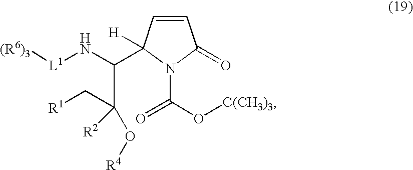

one of which is the compound having the absolute stereochemistry illustrated by a compound having structural formula (19-a)

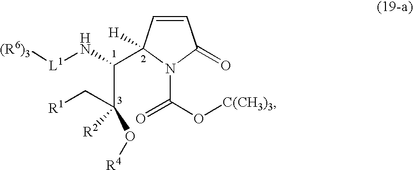

in which the substituents at the carbons labeled “1” and “2” are erythro relative to each other and “anti-” relative to the O—R⁴ group at the carbon labeled “3,”

a preferred embodiment of which is substantially pure, diastereomerically enriched tert-butyl (2R)-2-((1R,2S)-2-methoxy-2-methyl-1-((tritylsulfanyl)amino)pentyl)-5-oxo-2,5-dihydro-1-H1 -pyrrole-1 -carboxylate,

and the other of which is a compound having the absolute stereochemistry illustrated by a compound having structural formula (19-b)

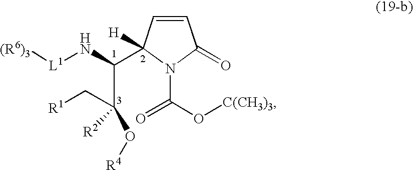

in which the substituents at the carbons labeled “1” and “2” are also erythro relative to each other but are “syn-” relative to the O—R⁴ group at the carbon labeled “3,”

a preferred embodiment of which is tert-butyl (2S)-2-((1S,2S)-2-methoxy-2-methyl-1-((tritylsulfanyl)amino)pentyl)-5-oxo-2,5-dihydro-1-1 H-pyrrole-1 -carboxylate, in which the (19-a)/(19-b) ratio in the mixture is about 4.3:1 to about 10:1;

(y) allowing the mixture of the compound having structural formula (19-a) and the compound having structural formula (19-b) to further equilibrate until the (19-a)/(19-b) ratio in the mixture is greater than 10:1;

and

(z) isolating the diastereomerically enriched compound having structural formula (19-a).

In a preferred sixth embodiment, steps (v) and (w) are conducted in situ, steps (x) and (y) are conducted sequentially, and steps (y) and (z) are conducted in situ.

Examples of fourth bases useful for the practice of step (v) are diethylmethylamine, diisopropylethylamine, dimethylbutylamine, 1,4-dimethylpiperazine, 1,8-diazobicyclo[5.4.0]undec-7-ene, 1,5-diazobicyclo[4.3.0]non-5-ene, 1-methylpiperidine, pyrazine, pyridazine, pyrimidine, pyridine, tetramethylenediamino-methane, tributylamine, triethylamine, tripropylamine, and the like.

A preferred fourth base for the practice of step (v) is triethylamine.

Step (v) is conducted at a temperature between about −10° C. and about 20° C. in a solvent such as carbon tetrachloride, chloroform, dichloromethane, dioxane, tetrahydrofuran, or a mixture thereof.

In a preferred embodiment, step (v) is conducted at about 0° C. in essentially tetrahydrofuran, after which the tetrahydrofuran is substantially replaced with heptane.

Examples of fifth bases useful for the practice of step (w) are diethylmethylamine, diisopropylethylamine, dimethylbutylamine, 1,4-dimethylpiperazine, 1,8-diazobicyclo[5.4.0]undec-7-ene, 1,5-diazobicyclo[4.3.0]non-5-ene, 1-methylpiperidine, pyrazine, pyridazine, pyrimidine, pyridine, tetramethylenediamino-methane, tributylamine, triethylamine, and tripropylamine, and the like.

A preferred fourth base for the practice of step (w) is triethylamine.

Step (w) is conducted at a temperature between about −10° C. and about 20° C. in a solvent such as carbon tetrachloride, chloroform, dichloromethane, dioxane, heptane, hexane, tetrahydrofuran, or a mixture thereof.

In a preferred embodiment, step (w) is conducted at about 0° C. in essentially heptane and dichloromethane, after which the dichloromethane is substantially removed.

Examples of second acids useful for the practice of step (x) are boron trifluoride.diethyletherate, copper(II) bis(trifluoromethanesulfonate), trifluoromethanesulfonic acid, titanium iso-propoxide, titanium tetrachloride, trimethylsilyltrifluoromethane sulfonate, tributylsilyltrifluoromethane sulfonate, ytterbium tetrakis(trifluoromethanesulfonate), zinc chloride, and the like.

A preferred second acid for the practice of step (x) is trifluoromethanesulfonic acid.

Step (x) is conducted at a temperature between about −50° C. and about −30° C. in a solvent such as acetonitrile, chloroform, carbon tetrachloride, dichloromethane, dioxane, heptane, hexane, tetrahydrofuran, or a mixture thereof.

In a preferred embodiment, step (x) is conducted at about −40° C. in essentially heptane and tetrahydrofuran.

Step (y) is conducted at a temperature between about −50° C. and about −30° C. in a solvent such as acetonitrile, chloroform, carbon tetrachloride, dichloromethane, dioxane, heptane, hexane, tetrahydrofuran, or a mixture thereof.

In a preferred embodiment, step (y) is conducted at about −40° C. in essentially heptane and tetrahydrofuran.

In a preferred embodiment of step (z), the tetrahydrofuran is substantially replaced with heptane to precipitate a substantially pure compound having structural formula (19-a), which is then isolated with a diastereomeric excess greater than about 90:1. In a more preferred embodiment of this step, the compound having structural formula (19-a) is isolated with a diastereomeric excess greater than about 99:1.

A compound having structural formula (19) is useful as an intermediate for making a compound having structural formula (24)

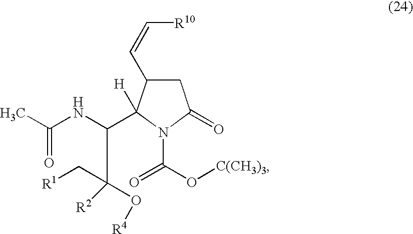

in which the carbon-carbon double bond is substantially in the Z configuration, and

in which R¹ is alkyl, cycloalkyl, cycloalkylalkyl, or arylalkyl;

R² is alkyl, cycloalkyl, cycloalkylalkyl, or arylalkyl;

R⁴ is alkyl, cycloalkyalkyl, or aryl-(C₂-C₄-alkyl); and

R¹⁰ is methyl, ethyl, iso-propyl, or vinyl.

In a seventh embodiment of this invention, therefore, is disclosed a process for making a compound having structural formula (24) from the compound having structural formula (19) comprising, in toto, the steps of:

(aa) reacting a compound having structural formula (19)

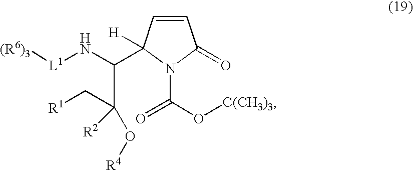

a preferred embodiment of which is a compound having the absolute stereochemistry illustrated by a compound having structural formula

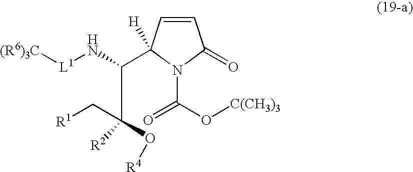

and a more preferred embodiment of which is substantially pure, diastereomerically enriched tert-butyl (2R)-2-((1R,2S)-2-methoxy-2-methyl-1-((tritylsulfanyl)amino)pentyl)-5-oxo-2, 5-dihydro-1-1H-pyrrole-1 -carboxylate,

a conjugate addition agent, said conjugate addition agent comprising a mixture of compounds having structural formula (20-Z)

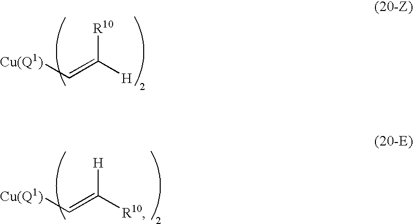

and structural formula (20-E)

in which the (20-Z)/(20-E) ratio in the mixture is about 97:3,

and in which Q¹ is Li, Mg—Cl, Mg—Br, or Mg—I,

and, optionally, a second additive at a temperature between about −50° C. and about −30° C. about to provide a compound having structural formula (21)

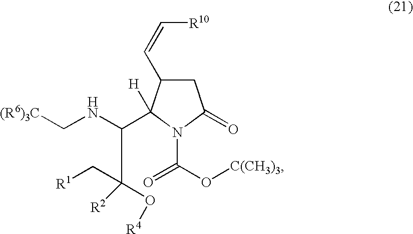

a preferred embodiment of which is a compound having the absolute stereochemistry illustrated by a compound having structural formula (21-a)

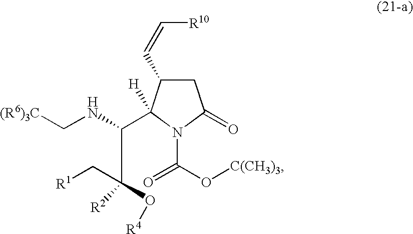

and a more preferred embodiment of which is substantially pure, diastereomerically enriched tert-butyl (2R,3S)-2-((1R,2S)-2-methoxy-2-methyl-1-((tritylsulfanyl)amino)pentyl)-5-oxo-3-((1Z)-1-propenyl)-1-pyrrolidinecarboxylate;

(bb) reacting the product of step (aa) and a third acid to provide a compound having the structural formula (22)

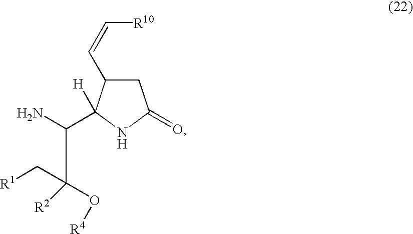

or a salt thereof,

a preferred embodiment of which is a compound having the absolute stereochemistry illustrated by a compound having structural formula (22-a)

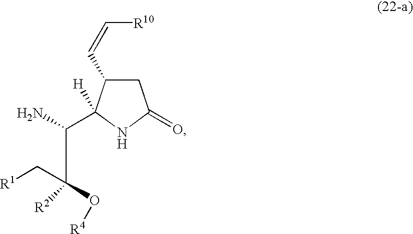

and a more preferred embodiment of which is substantially pure, diastereomerically enriched (4S,5R)-5-((1R,2S)-1-amino-2-methoxy-2-methylpentyl)-4-((1Z)-1-propenyl)-2-pyrrolidinone;

(cc) reacting the product of step (bb), an acetylating agent, and a sixth base to provide a compound having the structural formula (23)

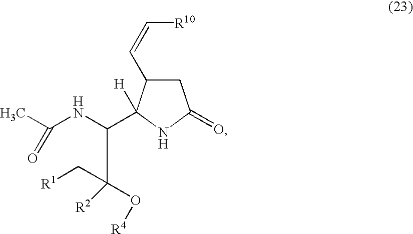

a preferred embodiment of which is a compound having the absolute stereochemistry illustrated by a compound having structural formula (23-a)

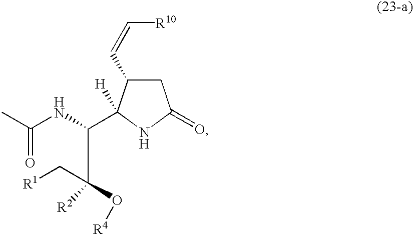

and a more preferred embodiment of which is substantially pure, diastereomerically enriched N-((1R,2S)-2-methoxy-2-methyl-1-((2R,3S)-5-oxo-3-((1Z)-1-propenyl)pyrrolidinyl)pentyl)acetamide;

(dd) reacting the product of step (cc), a tert-butylcarbonyloxy-introducing agent, a seventh base, and 4-dimethylaminopyridine to provide the compound having formula (24),

a preferred embodiment of which is a compound having the absolute stereochemistry illustrated by a compound having structural formula (24-a)

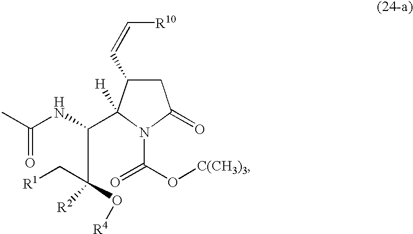

and a more preferred embodiment of which is substantially pure, diastereomerically enriched tert-butyl (2R,38)-2-((1R,2S)-1-(acetylamino)-2-methoxy-2-methylpentyl)-5-oxo-3-1-propenyl)-1-pyrrolidinecarboxylate;

and

(ee) optionally isolating the product of step (dd).

In a preferred seventh embodiment, steps (an), (bb), (cc), (dd), and (ee) are conducted in situ.

Examples of second additives useful for the practice of step (aa) are compounds having formula Si(R⁸)₃(Cl), in which each R⁸ is independently alkyl or unsubstituted aryl, compounds having formula Si(R⁸)₃(Br), and the like.

In a preferred embodiment for the practice of step (aa) the second additive is employed, is trimethylsilyl chloride, and is present in about 0.5 to about 4 molar equivalents per molar equivalent of the compound having structural formula (19). In a more preferred embodiment of this step, the second additive is present in about 3 molar equivalents per molar equivalent of the compound having structural formula (19).

In a preferred embodiment, step (aa) is conducted at about −25° C. in a solvent such as acetonitrile, chloroform, dichloromethane, ethyl acetate, iso-propyl acetate, tetrahydrofuran, toluene, or a mixture thereof.

In a more preferred embodiment, step (aa) is conducted at about −25° C. in essentially tetrahydrofuran and toluene.

In another part of the seventh embodiment of this invention, the conjugate addition agent used for the practice of step (aa) is made by reacting a mixture, said mixture comprising a precursor compound having structural formula (20-Z^{p})

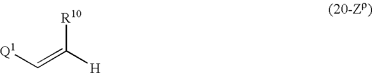

and a precursor compound having structural formula (20-E^{p})

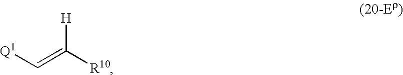

in which the (20-Z^{p})/(20-E^{p}) ratio in the mixture is about 97:3, and

in which Q¹ is Li, Mg—Cl, Mg—Br, or Mg—I, a cuprous adjuvant, and optionally, a ligand selected from the group comprising tri-n-butylphosphine, tri-iso-butylphosphine, and tri-n-pentylphosphine.

Examples of the cuprous adjuvant useful for the practice of step (aa) are copper(I) bromide, copper(I) bromide·dimethyl sulfide, copper(I) chloride, copper(I) bromide, copper(I) iodide, copper(I) cyanide, copper(I) tert-butoxide, copper(I) thiophenoxide, copper(I) thioisocyanate, copper(I) acetylide, copper(I) 3,3-dimethyl-1-butynide, copper(I) 1-pentynide, and the like.

In a preferred embodiment for the practice of step (aa), the ligand is omitted and the cuprous adjuvant is copper(I) bromide·dimethyl sulfide complex, which is present in about 0.05 molar equivalents to about 1.2 molar equivalents per molar equivalent of the compound having structural formula (19). In a more preferred embodiment of this step, the cuprous adjuvant is present in about 0.5 molar equivalents per molar equivalent of the compound having structural formula (19).

In still another part of the seventh embodiment of this invention, the 97:3 (20-Z)/(20-E) mixture used for the practice of step (aa), is present in about 0.9 molar equivalents to about 3.5 molar equivalents per molar equivalent of the compound having structural formula (19). In a more preferred embodiment of this step, the 97:3 (20-Z)/(20-E) mixture is present in about 3 molar equivalents per molar equivalent of the compound having structural formula (19).

Examples of third acids useful for the practice of step (bb) are hydrochloric acid, hydrobromic acid, sulfuric acid, phosphoric acid, trifluoroacetic acid, para-toluenesulfonic acid, pyridinium para-toluenesulfonic acid, camphorsulfonic acid, and the like.

In a preferred embodiment for the practice of step (bb), the third acid is hydrochloric acid.

Step (bb) is conducted at a temperature between about −10° C. and about −30° C. in a solvent such as ethyl acetate, iso-propyl acetate, tetrahydrofuran, toluene, water, or a mixture thereof.

In a preferred embodiment, step (bb) is conducted at a temperature of about −20° C. in essentially tetrahydrofuran, toluene, and water.

Examples of acetylating agents useful for the practice of step (cc) are acetic anhydride, acetyl chloride, acetyl bromide, and the like.

In a preferred embodiment for the practice of step (cc), the acetylating agent is acetic anhydride.

Examples of sixth bases for the practice of step (cc) are lithium hydroxide, sodium hydroxide, potassium hydroxide, lithium carbonate, sodium carbonate, potassium carbonate, cesium carbonate, sodium bicarbonate, potassium bicarbonate, diethylenediamine, diethylmethylamine, diisopropylethylamine, dimethylbutylamine, 1,4-dimethylpiperazine, 1,8-diazobicyclo[5.4.0]undec-7-ene, 1,5-diazobicyclo[4.3.0]non-5-ene, 1-methylpiperidine, pyrazine, pyridazine, pyrimidine, pyridine, tetramethylenediaminomethane, tributylamine, triethylamine, tripropylamine, and the like.

In a preferred embodiment for the practice of step (cc), the sixth base is sodium hydroxide.

Step (cc) is conducted at a temperature between about 0° C. and about 35° C. in a solvent selected from the group comprising chloroform, dichloromethane, tetrahydrofuran, toluene, water, or a mixture thereof.

In a preferred embodiment, step (cc) is conducted at about 25° C. in essentially water.

Examples of tert-butylcarbonyloxy-introducing agents useful for the practice of step (dd) are di-tert-butyl dicarbonate, 1-(tert-butoxycarbonyl)-2-tert-butyl-3-methyl-4-imidazolidinone, (2-tert-butoxycarbonyloxyimino)-2-phenylacetonitrile), and the like.

In a preferred embodiment for the practice of step (dd), the tert-butylcarbonyloxy-introducing agent is di-tert-butyl dicarbonate.

Examples of seventh bases useful for the practice of step (dd) are diethylmethylamine, diisopropylethylamine, dimethylbutylamine, 1,4-dimethylpiperazine, 1,8-diazobicyclo[5.4.0]undec-7-ene, 1,5-diazobicyclo[4.3.0]non-5-ene, 1-methylpiperidine, pyrazine, pyridazine, pyrimidine, pyridine, tetramethylenediaminomethane, tributylamine, triethylamine, tripropylamine, and the like.

In a preferred embodiment for the practice of step (dd), the seventh base is triethylamine.

Step (dd) is conducted at a temperature between about 0° C. and about 35° C. in a solvent such as acetonitrile, ethyl acetate, iso-propyl acetate, tetrahydrofuran, toluene, water, or a mixture thereof.

In a preferred embodiment, step (dd) is conducted at about 25° C. in essentially tetrahydrofuran.

In a preferred embodiment of step (ee), the tetrahydrofuran solution is mixed first with iso-propyl acetate, then with heptane, to crystallize the compound having structural formula (24).

A compound having structural formula (24) is useful as an intermediate for making a compound having structural formula (28)

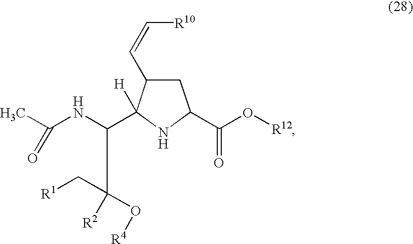

or a therapeutically acceptable salt thereof,

in which R¹ is alkyl, cycloalkyl, cycloalkylalkyl, or arylalkyl;

R² is alkyl, cycloalkyl, cycloalkylalkyl, or arylalkyl;

R⁴ is alkyl, cycloalkyalkyl, or aryl-(C₂-C₄-alkyl);

R¹⁰ is methyl, ethyl, iso-propyl, or vinyl; and

R¹² is hydrogen or alkyl.

In an eighth embodiment of this invention, therefore, is disclosed a process for making a compound having structural formula (28) from the compound having structural formula (24) comprising the steps of:

(ff) reacting a compound having structural formula (24)

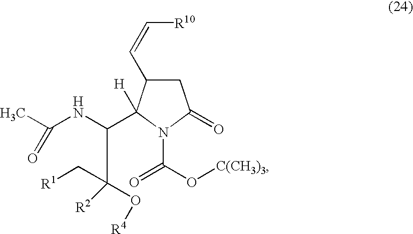

a preferred embodiment of which is a compound having the absolute stereochemistry illustrated by a compound having structural formula (24-a)

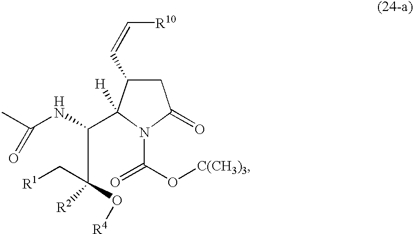

and a more preferred embodiment of which is substantially pure, diastereomerically enriched

tert-butyl (2R,3S)-2-((1R,2S)-1-(acetylamino)-2-methoxy-2-methylpentyl)-5-oxo-3-((1Z)-1-propenyl)-1-pyrrolidinecarboxylate,

and a fourth reducing agent to provide a compound having structural formula (25)

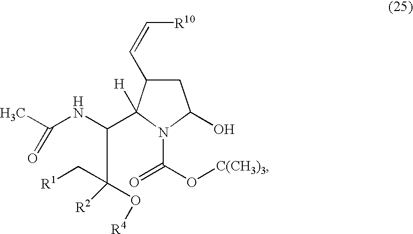

a preferred embodiment of which is a compound having the absolute stereochemistry illustrated by a compound having structural formula (25-a)

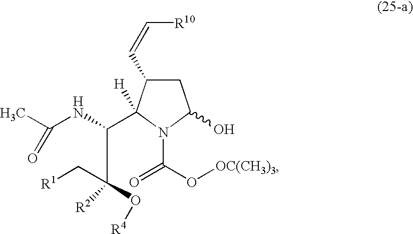

and a more preferred embodiment of which is substantially pure, diastereomerically enriched

2:1 anomeric tert-butyl (2R,3S)-2-((1R,2S)-1-(acetylamino)-2-methoxy-2-methylpentyl)-5-hydroxy-3-((1Z)- 1-propenyl)-1-pyrrolidinecarboxylate;

(gg) optionally reacting the product of step (ff), a C₁-C₅ alcohol, a trialkylorthoformate, and a second acid catalyst to provide a compound having structural formula (26)

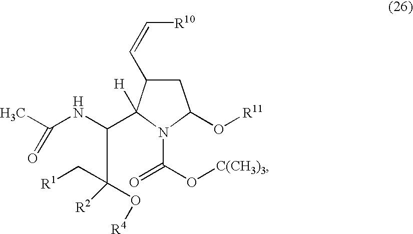

in which R¹¹ is methyl, ethyl, propyl, or iso-propyl,

a preferred embodiment of which is a compound having the absolute stereochemistry illustrated by a compound having structural formula (26-a)

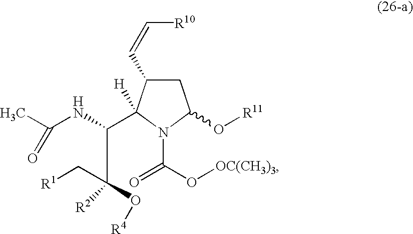

and a more preferred embodiment of which is substantially pure, diastereomerically enriched

### tert-butyl (2R,3S)-2-((1R,2S)-1-(acetylamino)-2-methoxy-2-methylpentyl)-5-methoxy-3-((1Z)-1-propenyl)-1-pyrrolidinecarboxylate;

(hh) reacting the product of step (ff) or step (gg), a cyanide-donating agent, and a fourth acid to provide a compound having structural formula (27)

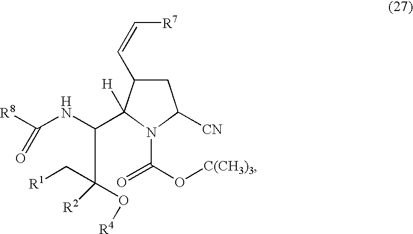

a preferred embodiment of which is a compound having the absolute stereochemistry illustrated by a compound having structural formula (27-a)

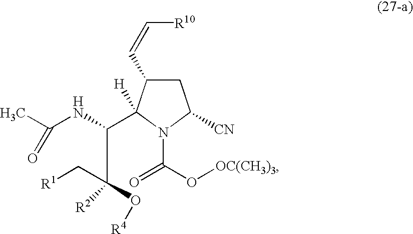

and a more preferred embodiment of which is substantially pure, diastereomerically enriched

tert-butyl (2R,3S,5R)-2-((1R,2S)-1-(acetylamino)-2-methoxy-2-methylpentyl)-((1Z)-1-propenyl)-1-pyrrolidinecarboxylate;

(ii) reacting the product of step (hh), a C₁-C₅ alcohol, and a fifth acid to provide the compound having structural formula (28),

a preferred embodiment of which is a compound having the absolute stereochemistry illustrated by a compound having structural formula (28-a)

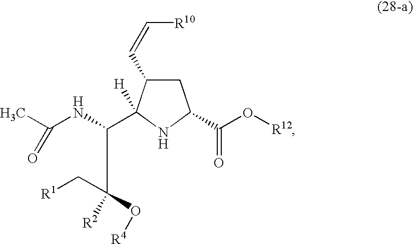

and a more preferred embodiment of which is substantially pure, diastereomerically enriched

isopropyl (2R,5R)-5-((1R,2S)-1-(acetylamino)-2-methoxy-2-methylpentyl)-4-((1Z)-1-propenyl-2-pyrrolidinecarboxylate,

or a therapeutically acceptable salt thereof, and

(jj) isolating the product of step (ii).

In a preferred eighth embodiment, step (gg) is conducted, and steps (ff), (gg), (hh), and (ii) are conducted in situ.

Examples of fourth reducing agents useful for the practice of step (ff) are lithium borohydride, sodium borohydride, potassium borohydride, lithium di(iso-butyl)aluminum hydride, lithium triethylborohydride, bis(2-methoxyethoxy)aluminum hydride, lithium tri-sec-butylborohydride, potassium tri-sec-butylborohydride, lithium aluminum hydride·N-methylpyrrolidine, and the like.

In a preferred embodiment for the practice of step (ff), the fourth reducing agent is lithium triethylborohydride.

Step (ff) is conducted at a temperature between about −20° C. and about 25° C. in a solvent such as chloroform, dibutylether, dichloromethane, diethylether, 1,2-dimethoxyethane, dioxane, hexane, heptane, tetrahydrofuran, toluene, or a mixture thereof.

In a preferred embodiment, step (ff) is conducted at about −15° C. to about 17° C. in essentially tetrahydrofuran.

Examples of C₁-C₃ alcohols useful for the practice of step (gg) are methanol, ethanol, propanol, iso-propanol, and the like.

In a preferred embodiment for the practice of step (gg), the C₁-C₃ alcohol is methanol.

Examples of trialkylorthoformates useful for the practice of step (gg) are trimethylorthoformate, triethylorthoformate, tripropylorthoformate, triisopropylorthoformate, and the like.

In a preferred embodiment for the practice of step (gg), the trialkylorthoformate is trimethylorthoformate.

Examples of second acid catalysts for the practice of step (gg) are pyridinium para-toluenesulfonate, para-toluenesulfonic acid, camphorsulfonic acid, sulfuric acid, methanesulfonic acid, and the like.

In a preferred embodiment for the practice of step (gg), the second acid catalyst is camphorsulfonic acid.

Step (gg) is conducted at a temperature between about 0° C. and about 30° C. in a solvent such as methanol, ethanol, n-propanol, and iso-propanol.

In a preferred embodiment, step (gg) is conducted at about 25° C. in essentially methanol.

Examples of cyanide-donating agents useful for the practice of step (hh) are sodium cyanide, trimethylsilylcyanide, tert-butyldimethylsilyl cyanide, zinc cyanide, diethylaluminum cyanide, and the like.

In a preferred embodiment for the practice of step (hh), the cyanide-donating agent is trimethylsilylcyanide.

Examples of fourth acids useful for the practice of step (hh) are aluminum chloride, boron trifluoride·diethyletherate, diethylaluminum chloride, magnesium bromide·diethyletherate tin(IV) chloride, titanium(IV) chloride, titanium(IV) tris(chloro)iso-propoxide, titanium(IV) bis(chloro)diisopropoxide, trifluoromethanesulfonic acid, tert-butyldimethylsilyl trifluoromethanesulfonate, triisopropylsilyl trifluoromethanesulfonate, trimethylsilyl trifluoromethanesulfonate, and triethylsilyl trifluoromethanesulfonate.

In a preferred embodiment for the practice of step (hh), the fourth acid is trifluoromethanesulfonic acid.

Step (hh) is conducted at a temperature between about −50° C. and about 0° C. in a solvent such as acetonitrile, dichloromethane, dibutylether, diethylether, tetrahydrofuran, toluene, or a mixture thereof.

In a preferred embodiment, step (hh) is conducted at about −40° C. in essentially acetonitrile.

Examples of fifth acids useful for the practice of step (ii) are hydrogen chloride/hydrochloric acid, hydrogen bromide/hydrobromic acid, sulfuric acid, trifluoromethanesulfonic acid, and the like.

In a preferred embodiment for the practice of step (ii), the fifth acid is hydrogen chloride/hydrochloric acid.

Step (ii) is conducted at a temperature beginning between about −50° C. and about −20° C. and ending between about 80° C. and about 90° C. in methanol, ethanol, propanol, iso-propanol, butanol, 1-methyl-1-butanol, 2-methyl-1-butanol, 1,1-dimethyl-1-butanol, pentanol, 1-methyl-1-butanol, 2-methyl-1-butanol, 3-methyl-1-butanol, 1-ethyl-1-propanol, or 2-ethyl-1-propanol.

In a preferred embodiment, step (ii) is conducted initially at about −30° C., then at about 25° C. for about one hour, then at about 90° C. for about eight hours in essentially iso-propanol.

In a preferred embodiment, step (jj) comprises isolating the compound having formula (28), or a therapeutically acceptable salt thereof, by crystallization.

In another part of the eighth embodiment of this invention, the compound having formula (28) is isopropyl (2R,5R)-5-((1R,2S)-1-(acetylamino)-2-methoxy-2-methylpentyl)-4-((1Z)-1-propenyl-2-pyrrolidinecarboxylate and is isolated by crystallization of its para-toluenesulfonate salt.

In still another part of the eighth embodiment of this invention, the isopropyl (2R,5R)-5-((1R,2S)-1-(acetylamino)-2-methoxy-2-methylpentyl)-4-((1Z)-1-propenyl)-2-pyrrolidinecarboxylate, para-toluenesulfonate is crystallized from iso-propyl acetate.

In still yet another part of the eighth embodiment of this invention, the crystallization comprises mixing a solution comprising essentially isopropyl (2R,5R)-5-((1R,2S)-1-(acetylamino)-2-methoxy-2-methylpentyl)-4-((l Z)-1-propenyl)-2-pyrrolidinecarboxylate in iso-propyl acetate and a solution comprising essentially para-toluenesulfonic acid monohydrate in iso-propyl acetate.

In still yet another part of the eighth embodiment of this invention, the crystallization of isopropyl (2R,5R)-5-((1R,2S)-1-(acetylamino)-2-methoxy-2-methylpentyl)-4-((1Z)-1-propenyl)-2-pyrrolidinecarboxylate, para-toluenesulfonate is conducted at a solution temperature of between about 40° C. and about 70° C., a preferred embodiment of which is between about 50° C. and about 60° C., in which the temperature of the solution is lowered to about 25° C. at a rate of about 5° C. per hour to precipitate the isopropyl (2R,5R)-5-((1R,2S)-1-(acetylamino)-2-methoxy-2-methylpentyl)-4-((1Z)-1-propenyl)-2-pyrrolidinecarboxylate, para-toluenesulfonate salt before isolating the same.

In still yet even another embodiment of this invention are disclosed compounds which are useful as intermediates for the preparation of neuraminidase inhibitors, said compounds having structural formula (29)

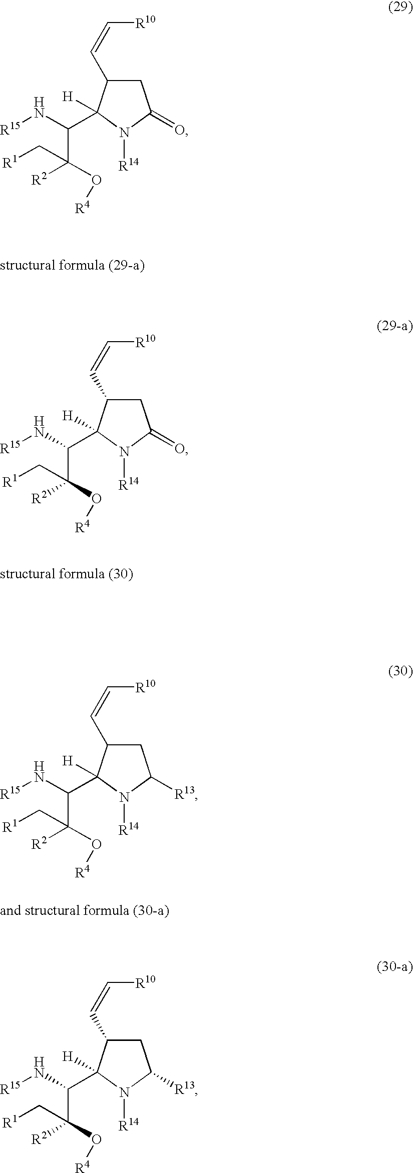

or salts thereof,

in which the carbon-carbon double bond in the compounds having structural formulas (29), (29-a), (30), and (30-a) is substantially in the Z configuration, and in which

R¹ is alkyl, cycloalkyl, cycloalkylalkyl, or arylalkyl;

R² is alkyl, cycloalkyl, cycloalkylalkyl, or arylalkyl;

R⁴ is alkyl, cycloalkyalkyl, or aryl-(C₂-C₄-alkyl);

R¹⁰ is methyl, ethyl, iso-propyl, or vinyl;

R¹³ is alkoxy alkoxycarbonyl, and hydroxy;

R¹⁴ is hydrogen or tert-butoxycarbonyl; and

R¹⁵ is hydrogen or acetyl,

with the proviso that R¹³ is alkoxycarbonyl only for the compound having structural formula (30-a),

preferred embodiments of which include

a compound having structural formula (29) or (29-a) in which R¹ is alkyl; R² is alkyl; R⁴ is alkyl; R¹⁴ is hydrogen, and R⁵ is hydrogen;

a compound having structural formula (29) or (29-a) in which R¹ is alkyl; R² is alkyl; R⁴ is alkyl; R¹⁴ is hydrogen, and R¹⁵ is acetyl;

a compound having structural formula (29) or (29-a) in which R¹ is alkyl; R² is alkyl; R⁴ is alkyl; R¹⁴ is tert-butoxycarbonyl, and R¹⁵ is acetyl;

a compound having structural formula (30) or (30-a) in which R¹ is alkyl; R² is alkyl; R⁴ is alkyl; R¹³ is alkoxy; R¹⁴ is tert-butoxycarbonyl, and R¹⁵ is hydrogen;

a compound having structural formula (30) or (30-a) in which R¹ is alkyl; R² is alkyl; R⁴ is alkyl; R¹³ is alkoxy; R¹⁴ is tert-butoxycarbonyl, and R¹⁵ is acetyl;

a compound having structural formula (30) or (30-a) in which R¹ is alkyl; R² is alkyl; R⁴ is alkyl; R¹³ is alkoxy; R¹⁴is hydrogen, and R¹⁵ is hydrogen;

a compound having structural formula (30) or (30-a) in which R¹ is alkyl; R² is alkyl; R⁴ is alkyl; R¹³ is alkoxy; R¹⁴ is hydrogen, and R¹⁵ is acetyl;

a compound having structural formula (30) or (30-a) in which R¹ is alkyl; R² is alkyl; R⁴ is alkyl; R¹³ is hydroxy; R¹⁴ is tert-butoxycarbonyl, and R¹⁵ is hydrogen;

a compound having structural formula (30) or (30-a) in which R¹ is alkyl; R² is alkyl; R⁴ is alkyl; R¹³ is hydroxy; R¹⁴ is tert-butoxycarbonyl, and R¹⁵ is acetyl;

a compound having structural formula (30) or (30-a) in which R¹ is alkyl; R² is alkyl; R⁴ is alkyl; R¹³ is hydroxy; R¹⁴ is hydrogen, and R¹⁵ is hydrogen;

a compound having structural formula (30) or (30-a) in which R¹ is alkyl; R² is alkyl; R⁴ is alkyl; R¹³ is hydroxy; R¹⁴ is hydrogen, and R¹⁵ is acetyl; and

a compound having structural formula (30-a) in which R¹ is alkyl; R² is alkyl; R⁴ is alkyl; R¹³ is alkoxycarbonyl; R¹⁴ is hydrogen, and R¹⁵ is acetyl, of which the more preferred embodiments of the foregoing are

the compound having structural formula (29) or (29-a) in which R¹ is alkyl; R² is alkyl; R⁴ is alkyl; R¹⁴ is hydrogen, and R¹⁵ is hydrogen;

the compound having structural formula (29) or (29-a) in which R¹ is alkyl; R² is alkyl; R⁴ is alkyl; R¹⁴ is hydrogen, and R¹⁵ is acetyl;

the compound having structural formula (29) or (29-a) in which R¹ is alkyl; R² is alkyl; R⁴ is alkyl; R¹⁴ is tert-butoxycarbonyl, and R¹⁵ is acetyl;

the compound having structural formula (30) or (30-a) in which R¹ is alkyl; R² is alkyl; R⁴ is alkyl; R¹³ is alkoxy; R¹⁴ is tert-butoxycarbonyl, and R¹⁵ is acetyl;

the compound having structural formula (30) or (30-a) in which R¹ is alkyl; R² is alkyl; R⁴ is alkyl; R¹³ is hydroxy; R¹⁴ is tert-butoxycarbonyl, and R¹⁵ is acetyl; and

the compound having structural formula (30-a) in which R¹ is alkyl; R² is alkyl; R⁴ is alkyl; R¹³ is alkoxycarbonyl; R¹⁴ is hydrogen, and R¹⁵ is acetyl, and more preferred embodiments of which include

a compound having structural formula (29) or (29-a) which is (4S,5R)-5-((1R,2S)-1-amino-2-methoxy-2-methylpentyl)-4-((1Z)-1-propenyl)-2-pyrrolidinone;

a compound having structural formula (29) or (29-a) which is N-((1R,2S)-2-methoxy-2-methyl-1-((2R,3S)-5-oxo-3-((1Z)-1-propenyl)pyrrolidinyl-pentyl)acetamide;

a compound having structural formula (29) or (29-a) which is tert-butyl (2R,3S)-2-((1R,2S)-1-(acetylamino)-2-methoxy-2-methylpentyl)-5-oxo-3-((1Z)-1-propenyl)-1-pyrrolidinecarboxylate;

a compound having structural formula (30) or (30-a) which is anomeric tert-butyl (2R,3S)-2-((1R,2S)-1-(acetylamino)-2-methoxy-2-methylpentyl)-5-hydroxy-3-((1Z)-1-propenyl)-1-pyrrolidinecarboxylate;

a compound having structural formula (30) or (30-a) which is tert-butyl (2R,3S)-2-((1R,2S)-1-(acetylamino)-2-methoxy-2-methylpentyl)-5-methoxy-3-((1Z)-1-propenyl)-1-pyrrolidinecarboxylate; and

a compound having structural formula (30-a) which is isopropyl (2R,5R)-5-((1R,2S)-1-(acetylamino)-2-methoxy-2-methylpentyl)-4-((1Z)-1- propenyl)-2-pyrrolidinecarboxylate, para-toluenesulfonic acid salt.

It is meant to be understood that the term “substantially pure” means greater than 90% pure; preferably greater than 95% pure, and more preferably greater than 99% pure.

It is also meant to be understood that the term “essentially,” in reference to a solvent or solvents, means greater than 90% of that solvent or solvents; preferably greater than 95% of that solvent or solvents, and more preferably greater than 99% of that solvent or solvents.

It is also meant to be understood that many of the compounds of this invention contain one chiral carbon atom and exist as enantiomers in the (R) or (S) configuration or as racemic mixtures comprising varying amounts of one enantiomer over the other. Mixtures comprising equimolar amounts of enantiomers are racemic mixtures and are designated (±). Mixtures comprising varying amounts of one enantiomer over the other are designated enantiomerically enriched with the enantiomer present in the higher amount. The enantiomeric enrichment of a mixture can be determined by gas or liquid chromatography in a chiral environment such as, for example, a column packed with a chiral auxiliary. Preferred enantiomeric enrichment for the purpose of this invention is about 86-89% enantiomeric excess of one enantiomer over the other. A more preferred enantiomeric enrichment for the purpose of this invention is about 94-97% enantiomeric excess of one enantiomer over the other. A still more preferred enantiomeric enrichment for the purpose of this invention is a substantially enantiomerically enriched mixture in which there is greater than a 99% enantiomeric excess of one enantiomer over the other.

It is also meant to be understood that many of the compounds of this invention which contain more than one chiral carbon atom exist as diastereomers. Diastereomeric enrichment or diastereomeric excess is determined by the enantiomeric excess of each chiral carbon atom in the diastereomer. Absolute stereochemistry is determined by the spatial orientation of each enantiomeric center relative to the other. The spatial orientation of each substituent relative to the other in a diastereomer can be predicted with a high degree of certainty by employing a substantially enantiomerically enriched starting materials such as, for example, the synthesis employing substantially enantiomerically enriched compound having formula (15-a) in step (x), also recited in the sixth embodiment of this invention. Preferred diastereomeric enrichment for the purpose of this invention is an isolated yield of about 93:7 to about 95:5 mixture of diastereomers. A more preferred diastereomeric enrichment for the purpose of this invention is an isolated yield of about 99.1:0.9 mixture of diastereomers. Because the diastereomers of this invention have different solubilities, they can be separated by column chromatography on silica gel as described in the '787 application or, more preferably, by crystallization.

It is also to be understood that the compound of this invention having structural formula (25) exists as anomers in which the chiral carbon atom of the pyrrolidine ring bearing the hydroxy group exists as an equilibrium mixture of (R) and (S) isomers at that chiral carbon atom.

It is also meant to be understood that many of the compounds of this invention contain a carbon-carbon double bond and can therefore exist as geometric isomers. This invention contemplates the various geometric isomers and mixtures thereof resulting from the arrangement of substituents around a carbon-carbon double bond and designates such isomers as being in the Z or E configuration, in which the term “Z” represents the larger two of the four substituents disposed on same side of a carbon-carbon double bond and the term “E” represents the larger two of the four substituents disposed on opposite sides of a carbon-carbon double bond. Preferred geometric isomeric enrichment for the purpose of this invention is a ratio greater than about 87:13 Z/E. More preferred geometric isomeric enrichment for the purpose of this invention is a ratio of about 98.8:1.2 Z/E. A still more preferred geometric isomeric enrichment for the purpose of this invention is a ratio of about 99.3:0.7 Z/E. Such preferred geometrically isomerically enriched compounds are defined as being substantially in the Z configuration.

It is also meant to be understood that the compounds of this invention can exist as therapeutically acceptable salts. The term “therapeutically acceptable salt” means salts or zwitterionic forms of the compounds which are water or oil-soluble or dispersible, which are suitable for inhibition of neuraminidases without undue toxicity, irritation, and allergic response, which are commensurate with a reasonable benefit/risk ratio, and which are effective for their intended use. The salts can be prepared during the final isolation and purification of a compound having structural formula (28) by derivatization of the same as a salt such as citrate, aspartate, benzoate, benzenesulfonate, bisulfate, butyrate, camphorate, camphorsufonate, digluconate, formate, fumarate, glycerophosphate, hemisulfate, heptanoate, hexanoate, hydrochloride, hydrobromide, hydroiodide, lactate, maleate, mesitylenesulfonate, methanesulfonate, naphthylenesulfonate, nicotinate, oxalate, pamoate, pectinate, persulfate, 3-phenylpropionate, picrate, pivalate, propionate, succinate, tartrate, thiocyanate, trichloroacetic, trifluoroacetic, phosphate, glutamate, bicarbonate, para-toluenesulfonate, undecanoate, and the like.

In a preferred embodiment for the practice of this invention, the compound having structural formula (28) is prepared as the para-toluenesulfonate salt.

The invention will now be described in connection with certain specific embodiments which are not intended to limit its scope. On the contrary, the invention covers all alternatives, modifications, and equivalents which are included within the scope of the claims. Thus, the following examples will illustrate an especially preferred practice of the invention, it being understood that the examples are for the purposes of illustration of certain preferred embodiments and are presented to provide what is believed to be the most useful and readily understood description of its procedures and conceptual aspects.

### EXAMPLE 1

### methyl (2E)-2-methyl-2-pentenoate

A solution of (2E)-2-methyl-2-pentenoic acid (1027 g), triethylformate (1031 g), and concentrated H₂SO₄ (24 mL) in methanol (3.23L) was stirred at reflux for 10 hours, cooled to ambient temperature, and treated with NaHCO₃ (76 g). The methanol layer was isolated and distilled at 45 mm Hg to provide fractions which distilled at 38-42° C. and 68-72° C., the latter of which provided 820 g of desired product. The 38-42° C. fraction was concentrated to one-quarter of its original volume and redistilled to provide an additional 200 g of the desired product (88.5% total) which azeotropes with methanol.

¹H NMR (CDCl₃) δ 6.8−6.72 (m, 1H), 3.73 (s, 3H), 2.5−2.2 (m, 2H), 1.83 (m, 3H) 1.04 (t, J=5.1 Hz, 3H).

### EXAMPLE 2

### (2E)-2-methyl-2-penten-1-ol

A solution of 1M diisobutylaluminum hydride in hexane (2.768 Kg) at −60° C. was treated with Example 1 (230 g) over 45 minutes, stirred for 1 hour, warmed to ambient temperature, added to a slurry of ice (1.3 Kg) and 50% (w/w) aqueous NaOH (1.3 Kg) over 1 hour, stirred for 2 hours at 0° C., and warmed to ambient temperature over 18 hours. The hexane layer was isolated and concentrated, and the concentrate was fractionally distilled at 50 mm Hg to provide 160 g (89%) of the desired product as a fraction which distilled at 82-84° C. ¹H NMR (CDCl₃) δ 5.44−5.38 (m, 1H), 3.96 (d, J=5.42 Hz, 2H), 2.1−2.0 (m, 2H), 1.65 (s, 3H), 0.96 (t, J=5.09 Hz, 3H).

### EXAMPLE 3

### 3,4-anhydro-1,2-dideoxy-4-methyl-D-threo-pentitol

A slurry of powdered 4 Å sieves (81 g) in dichloromethane (2.7L) at −20° C. was treated sequentially with dimethyl D-tartrate (102.4 g), titanium iso-propoxide (159 g), and Example 2 (180 g) in dichloromethane (315 mL), stirred for 20 minutes, cooled to −40° C., treated with pre-dried (4 Å sieves) 5-6M tert-butyl hydroperoxide in decane (719 mL), stirred for 2 hours, treated with dimethyl sulfide (90 mL) and 30% (w/w) aqueous NaOH (750 mL), stirred for 2 hours, warmed to 15° C., and treated with water (1L). The aqueous layer was isolated and extracted with dichloromethane (1L). The organics were combined and concentrated to provide 104 g (50%) of the desired product (and some decane), which was used directly in the next step. ¹H NMR (CDCl₃) δ 3.8−3.6 (m, 1H), 3.0 (t, J=6.3 Hz, 2H), 1.7 (m, 1H), 1.7−1.53 (m, 2H), 1.29 (s, 3H), 1.04 (t, J=7.8 Hz, 3H).

### EXAMPLE 4

### (2S)-2-methyl-1,2-pentanediol

A solution of 1M lithium aluminum hydride in THF (3.59L) at −40° C. was treated with a solution of Example 3 (104 g) in THF (300 mL) over 2 hours, warmed to ambient temperature over 18 hours, cooled to −20° C., treated sequentially with water (100 mL), 15% (w/w) aqueous NaOH (100 mL), and water (150 mL), each over 1 hour, stirred for 2 hours while warming to 10° C., filtered through diatomaceous earth (Celite®), and concentrated. The concentrate was fractionally distilled at 45 mm Hg to provide 105.1 g (50%, two steps) of the desired product (85-93% ee) as a fraction which distilled at 128-130° C. ¹H NMR (CDCl₃) δ 3.5−3.38 (m, 2H), 1.5−1.31 (m, 4H), 1.17 (s, 3H), 0.94 (t, J=7.1 Hz, 3H).

### EXAMPLE 5

### ((((2S)-2-methoxy-2-methylpentyl)oxy)methyl)benzene

### (2E)-2-methyl-2-penten-1-ol

A solution of 1M diisobutylaluminum hydride in hexane (2.768 Kg) at −60° C. was treated with Example 1 (230 g) over 45 minutes, stirred for 1 hour, warmed to ambient temperature, added to a slurry of ice (1.3 Kg) and 50% (w/w) aqueous NaOH (1.3 Kg) over 1 hour, stirred for 2 hours at 0° C., and warmed to ambient temperature over 18 hours. The hexane layer was isolated and concentrated, and the concentrate was fractionally distilled at 50 mm Hg to provide 160 g (89%) of the desired product as a fraction which distilled at 82-84° C. ¹H NMR (CDCl₃) δ 5.44−5.38 (m, 1H), 3.96 (d, J=5.42 Hz, 2H), 2.1−2.0 (m, 2H), 1.65 (s, 3H), 0.96 (t, J=5.09 Hz, 3H).

### EXAMPLE 3

### 3,4-anhydro-1,2-dideoxy-4-methyl-D-threo-pentitol

A slurry of powdered 4 Å sieves (81 g) in dichloromethane (2.7L) at −20° C. was treated sequentially with dimethyl D-tartrate (102.4 g), titanium iso-propoxide (159 g), and Example 2 (180 g) in dichloromethane (315 mL), stirred for 20 minutes, cooled to −40° C., treated with pre-dried (4 Å sieves) 5-6M tert-butyl hydroperoxide in decane (719 mL), stirred for 2 hours, treated with dimethyl sulfide (90 mL) and 30% (w/w) aqueous NaOH (750 mL), stirred for 2 hours, warmed to 15° C., and treated with water (1L). The aqueous layer was isolated and extracted with dichloromethane (1L). The organics were combined and concentrated to provide 104 g (50%) of the desired product (and some decane), which was used directly in the next step. ¹H NMR (CDCl₃) δ 3.8−3.6 (m, 1H), 3.0 (t, J=6.3 Hz, 2H), 1.7 (m, 1H), 1.7−1.53 (m, 2H), 1.29 (s, 3H), 1.04 (t, J=7.8 Hz, 3H).

### EXAMPLE 4

### (2S)-2-methyl-1,2-pentanediol

A solution of 1M lithium aluminum hydride in THF (3.59L) at −40° C. was treated with a solution of Example 3 (104 g) in THF (300 mL) over 2 hours, warmed to ambient temperature over 18 hours, cooled to −20° C., treated sequentially with water (100 mL), 15% (w/w) aqueous NaOH (100 mL), and water (150 mL), each over 1 hour, stirred for 2 hours while warming to 10° C., filtered through diatomaceous earth (Celite®), and concentrated. The concentrate was fractionally distilled at 45 mm Hg to provide 105.1 g (50%, two steps) of the desired product (85-93% ee) as a fraction which distilled at 128-130° C. ¹H NMR (CDCl₃) δ 3.5−3.38 (m, 2H), 1.5−1.31 (m, 4H), 1.17 (s, 3H), 0.94 (t, J=7.1 Hz, 3H).

### EXAMPLE 5

### ((((2S)-2-methoxy-2-methylpentyl)oxy)methyl)benzene

A suspension of dry sodium hydride (prepared by washing 6.18 g of 60% oily sodium hydride with heptane (2×200 mL)) in THF at 0° C. was treated sequentially with a solution of Example 4 (82.4 g) in THF (50 mL) over 30 minutes and benzyl bromide (90.6 mL) over 30 minutes, stirred for 12 hours at ambient temperature, cooled to 0° C., treated with methyl iodide (272 mL), stirred for 18 hours at ambient temperature, and treated with water (500 mL) and methyl tert-butyl ether (500 mL). The organic layer was isolated and concentrated. The concentrate was treated sequentially with with N,N-dimethyl ethylenediamine (18.5 g), water (1L) and methyl tert-butyl ether (1L). The organic layer was isolated, washed with 1M HCl (100 mL) and 3% (w/w) aqueous sodium chloride (200 mL) and concentrated to provide 145 g (93.5%) of the desired product, which was used directly in the next step. ¹H NMR (CDCl₃) δ 7.35−7.26 (m, 5H), 4.55 (s, 2H), 3.32 (dd, J=9.8 and 12.5 Hz, 2H), 3.22 (s, 3H), 1.58=1.42 (m, 2H), 1.34−1.22 (m, 2), 1.14 (s, 3H), 0.9 (t, J=7.1 Hz, 3H).

### EXAMPLE 6

### (2S)-2-methoxy-2-methyl-1-pentanol

A solution of Example 5 (99.5 g) in THF (650 mL) at ambient temperature was treated with 5% palladium hydroxide on carbon (13 g), stirred under hydrogen at 40 pounds per square inch for 18 hours, and filtered through diatomaceous earth (Celite®). The filtrate was treated with 5% palladium hydroxide on carbon (13 g), stirred under hydrogen at 40 pounds per square inch for 1 hour, filtered through diatomaceous earth (Celite®), and concentrated. The concentrate was fractionally distilled at 50 mm Hg, and fractions which distilled at 80-90° C. and 120-140° C. were collected and combined to provide 48.9 g (82.7%) of the desired product and some toluene. ¹H NMR (CDCl₃) δ 3.55 (q, J=11.1 Hz, 2H), 3.3 (s, 3H), 1.6−1.44 (m, 4H), 1.2 (s, 3H), 1.03 (t, J=7.4 Hz, 3H).

Examples 7-10 describe an alternate synthesis of Example 6.

### EXAMPLE 7

### 2-methoxy-2-methylpentanoic acid

A solution of 2-pentanone (2 Kg) and bromoform (61 Kg) in methanol (6.3 Kg) at 0° C. with overhead stirring was treated with a solution of potassium hydroxide (10.6 Kg) in methanol (19 Kg) over 5 hours, filtered, and concentrated. The concentrate was treated with 20% (w/w) aqueous sodium hydroxide to pH 9. The aqueous layer was isolated, stirred at 50° C. for 2 hours, cooled to ambient temperature, washed with methyl tert-butyl ether (3L), cooled to 5° C., acidified with concentrated HCl to pH 2-3, and extracted with methyl tert-butyl ether (18L). The extract was washed with 10% (w/w) aqueous sodium chloride (3L) and concentrated to provide 2.6 Kg (77%) of the desired product which was used directly in the next step. ¹H NMR (CDCl₃) δ 11.6 (s, 1H), 3.26 (s, 3H), 1.39 (s, 3H), 1.8−1.23 (m, 4H), 0.89 (t, 3H, J=7.0 Hz).

### EXAMPLE 8

### methyl 2-methoxy-2-methylpentanoate

A solution of Example 7 (2.5 Kg) in methanol (12L) at ambient temperature was treated with concentrated sulfuric acid (200 mL), heated to reflux for 16 hours, cooled to ambient temperature, treated with sodium bicarbonate (1.5 Kg), and concentrated. The concentrate was dissolved in methyl tert-butyl ether (8L), extracted with water (4L) and saturated aqueous Na₂CO₃ (100 mL), and concentrated. The concentrate was distilled at 50 mm Hg, and a fraction boiling at 90-95° C. was collected to provide 2.1 Kg (77%) of the desired product. ¹H NMR (CDCl₃) δ 3.7 (s, 3H), 3.25 (s, 3H), 1.7−1.6 (m, 2H), 1.35 (s 3H), 1.3−1.15 (m, 2H), 0.89 (t, 3H, J=7.0 Hz).

### EXAMPLE 9

### (2S)-2-methoxy-2-methylpentanoic acid

Candida Ruglosa (20 g, 875 units/mg) was treated sequentially with 0.05M phosphate buffer (pH 8) and a solution of Example 8 (20 g) in acetone (50 mL), heated at 37° C. for 116 hours with a pH adjustment to 4.5-6 with 2M NaOH after 74 hours, acidified to pH 2 with 2M HCl, and extracted with ethyl acetate (500 mL). This extract was extracted with 10% (w/w) aqueous Na₂CO₃ (200 mL and 100 mL), and the combined aqueous extracts were acidified to pH 3 with concentrated HCl and extracted with methyl tert-butyl ether (300 mL). This extract was dried (Na₂SO₄), filtered, and concentrated to provide 8.5 g (93% recovery, >99% ee) of the desired product, which was used directly in the next step. ¹H NMR (CDCl₃) δ 3.43 (q, J=12 Hz, 2H), 3.2 (s, 3H), 1.52−1.4 (m, 2H), 1.22−1.1 (m, 2H), 1.18 (s, 3H), 0.94 (s, 3H).

### EXAMPLE 10

### (2S)-2-methoxy-2-methyl-1-pentanol

A solution of Example 9 (507 g) in dichloromethane (3L) at 0° C. was treated with 10M borane-dimethyl sulfide (624 mL), stirred for 5 hours at ambient temperature, cooled to 0° C. to 5° C., treated with 2M NaOH (3.3L), warmed to ambient temperature and stirred for several hours. The layers were separated, and the water layer was extracted with dichloromethane (2×1.6L). The combined extracts were washed with 2M NaOH (1.65L) and 10% (w/w) aqueous NaCl (1.65L), dried (MgSO₄), filtered, and concentrated to provide 460 g (100%) of the desired product, which was used directly in the next step. ¹H NMR (CDCl₃) δ 3.55 (q, J=11.1 Hz, 2H), 3.3 (s, 3H), 1.6−1.44 (m, 4H), 1.2 (s, 3H), 1.03 (t, J=7.4 Hz, 3H).

### EXAMPLE 11

### (2S)-2-methoxy-2-methylpentanal

A solution of Example 8 (19.21 g) and triethylamine (60 mL) in dichloromethane (40 mL) at 0° C. was treated with sulfur trioxide-pyridine (35 g) in DMSO (160 mL) over 2.5 hours, stirred for 30 minutes, and treated with 2M H₃PO₄ (325 mL) over 30 minutes. The aqueous layer was isolated and extracted with dichloromethane (160 mL), and the combined organics were washed with 2M H₃PO₄ (325 mL), dried (flash silica), filtered, and concentrated with intermittent dichloromethane treatment to provide 69.43 g of a 29.7% (w/w) solution of the desired product (97.8%), which was used directly in the next step. ¹H NMR (CDCl₃) δ 9.58 (s, 1H), 3.28 (s, 3H), 1.58 (m, 2H), 1.29 (m, 2H), 1.22 (s, 3H), 0.92 (t, J=7.4 Hz, 3H).

### EXAMPLE 12

### (1E,2S)-2-methoxy-2-methylpentanal S-tritylthioxime

The solution of the 29.7% (w/w) solution of Example 11 in dichloromethane (60.24 g) at ambient temperature was treated sequentially with triphenylmethanesulfenamide (39.03 g), pyridinium para-toluenesulfonate (688 mg), and sodium sulfate (9.81 g), stirred for 18 hours, treated with heptane (32 mL), stirred for 5 minutes, and filtered. The filtrate was concentrated with intermittent heptane treatment to provide 80 g of a 64% (w/w) solution of the desired product in heptane. This solution was filtered through a silica gel plug (prepared from flash silica gel (64 g) and heptane in a 150 mL filter funnel with a 9 cm diameter and 3 cm height) with 5% methyl tert-butyl ether/heptane (512 mL). The filtrate was concentrated to provide 101.9 g of a solution of 50% (w/w) of the substantially enantiomerically enriched desired product (93%), which was used directly in the next step. ¹H NMR (CDCl₃) δ 7.56 (s, 1H), 7.28 (m, 15H), 2.96 (s, 3H), 1.35 (m, 2H), 1.16 (m, 2H), 0.80 (t, J=7.4 Hz).

### EXAMPLE 13

### tert-butyl-4-hydroxy-2-oxo-pyrrolidinecarboxylate

A solution of 4-amino-3-hydroxy-n-butyric acid (25 g) in pyridine (125 mL) was treated with hexamethyldisilazane (71.5 mL) and xylenes (250 mL), stirred at reflux for 16 hours, concentrated to 50 mL, cooled to 3° C., treated sequentially with 4-dimethylaminopyridine (1.22 g) in THF (40 mL) and di-tert-butyldicarbonate (54.96 g) in THF (80 mL), stirred for 14 hours at ambient temperature, treated with triethylamine trihydrofluoride (15 mL), stirred for 2 hours, treated with heptane, cooled to 3° C., and filtered. The filtrant was washed with heptane (150 mL) and dried under vacuum at 50° C. to provide 41 g (97%) of the desired product. ¹H NMR (CDCl₃) δ 4.37 (m, 1H), 3.65 (dd, J=5.5, 11.4 Hz, 1H), 3.63 (ddd, J=1.1, 3.3, 11.8 Hz, 1H), 2.71 (dd, J=6.2, 17.3 Hz, 1H), 2.47 (ddd, J=1.1, 3.6, 17.2 Hz), 1.53 (s, 9H).

### EXAMPLE 14

### tert-butyl 2-oxo-3-pyrroline carboxylate

A solution of Example 13 (3.2 Kg) in THF (31.26 Kg) was treated with triethylamine (4.425 Kg), cooled to 0.8° C., treated with methanesulfonyl chloride (2.32 Kg) over 2 hours, stirred for an additional 2 hours, treated with ethyl acetate (40L), and filtered. The filtrate was washed with 15% (w/w) aqueous NaCl (2×13L), and the combined washings were extracted with isopropyl acetate (13.3L). The filtrant was rinsed with isopropyl acetate (13.3L), and the organics were combined and concentrated to 20L with intermittent heptane treatment (4×20L) to keep its volume at 30L during distillation. The concentrate was treated with dichloromethane (12L) to provide a solution of 2.5369 Kg of the desired product (88.6%), which was used directly in the next step. ¹H NMR (CDCl₃) δ 7.18 (dt, J=6.2, 1.9 Hz, 1H), 6.16 (dt, J=6.2, 1.9 Hz, 1H), 4.35 (t, J=1.9, 2H), 1.56 (s, 9H).

### EXAMPLE 15

### tert-butyl 2-((tert-butyl(dimethyl)silyl)oxy)-1H-pyrrole-1-carboxylate

Diatomaceous earth (Celite®, 3.3 Kg) was treated sequentially with the solution of Example 14 (2.506 Kg), dichloromethane (25L), and heptane (25.8L). The resulting solution/slurry was treated with triethylamine (4.5 Kg), cooled to 0° C., and treated with tert-butyldimethylsilyl trifluoromethanesulfonate (4.71 Kg) over 2 hours, stirred for 1 hour, treated with methanol (1L) over 10 minutes, stirred for an additional 20 minutes, concentrated to 20L with intermittent heptane treatment (3×20L), filtered, concentrated to 7.913 Kg, and treated with heptane (3.5 Kg) to provide 11.41 Kg of a 39.3% (w/w) solution of the desired product (96.5%), which was used directly in the next step. ¹H NMR (CDCl₃) δ 6.69 (dd, J=2.2, 4.0 Hz, 1H), 5.90 (dd, J=3.7, 4.0, 1H), 5.23 (dd, J=2.2, 3.7 Hz, 1H), 1.57 (s, 9H), 0.99 (s, 9H), 0.22 (s, 6H).

### EXAMPLE 16

### tert-butyl (2R)-2-((1R,2S) 2-methoxy-2-methyl-1-((tritylsulfanyl)amino)pentyl)-5-oxo-2,5-dihydro-1-1H-pyrrole-1-carboxylate

A solution of 44.8% (w/w) of Example 12 in heptane (2.534 Kg) was treated with the solution of 39.9% (w/w) of Example 15 in heptane (3.068 Kg) and THF (14.54 Kg), cooled to −40° C., and treated with trifluoromethanesulfonic acid (320 g) over 40 minutes with additional trifluoromethanesulfonic acid (11 mL, 5.5 mL, and 22 mL) added at 1, 2, and 4.5 hour intervals, respectively. After the last addition, the solution was stirred for 1 hour at −40° C., washed with 0.5M NaHCO₃ (16.3 Kg) and brine (3.8 Kg), treated with heptane (3.15 Kg), and concentrated with intermittent heptane treatment (3.2 Kg, 4.3 Kg, 1.6 Kg, and 5.6 Kg) to keep its volume at 8L during the distillation. A solid that precipitated from the mother liquor during the distillation was filtered and dried under vacuum at 50° C. to provide 1.353 Kg (82%) of the diastereomerically enriched desired product. ¹H NMR (CDCl₃) δ 7.22 (dd, J=2.2, 6.2 Hz, 1H), 7.14 (m, 15H), 5.92 (dd, J=1.5, 6.3 Hz, 1H), 4.73 (m, 1H), 3.76 (dd, J=2.9, 11.4 Hz, 1H), 2.95 (s, 3H), 2.52 (d, J=11.4 Hz, 1H), 1.47 (m, 3H), 1.26 (s, 9H), 1.20 (m, 3H), 0.98 (m, 1H), 0.82 (t, J=6.6 Hz, 3H).

### EXAMPLE 17

### tert-butyl (2R,3S)-2-((1R,2S)-2-methoxy-2-methyl-1-((tritylsulfanyl)amino)pentyl)-5-oxo-3-((1Z)-1-propenyl)-1-pyrrolidinecarboxylate

A suspension of copper(I) bromide·dimethylsulfide complex (8.74 g) in THF (750 mL) at −30° C. was treated with 0.485M 1-propenyl magnesium bromide in THF (526 mL) over 15 minutes and trimethylsilyl chloride (11.09 g) over 5 minutes, stirred for another 15 minutes, warmed to −25° C., treated with Example 16 (50 g) in toluene (750 mL) over 20 minutes, stirred for another 5 minutes, treated with a solution comprising water (75 mL), concentrated NH₄OH (75 mL), and 25% (w/w) aqueous NH₄Cl (600 mL), warmed to ambient temperature, and stirred for 1 hour. The organic layer was isolated and washed with a solution comprising water (75 mL), concentrated NH₄OH (75 mL), 25% (w/w) and aqueous NH₄Cl (600 mL), and then with brine (600 mL) to provide a solution of the desired product, in substantially in the Z configuration, which was used directly in the next step. ¹H NMR (CDCl₃) δ 7.33−7.15 (m, 15H), 5.38 (m, 2H), 3.99 (d, J=1.4 Hz, 1H), 3.71 (t, J=9.2 Hz, 1H), 3.59 (dd, J=2.2, 10.3 Hz, 1H), 3.06 (d, J=10.6 Hz, 1H), 3.03 (s, 3H), 2.81 (dd, J=9.6, 17.6 Hz, 1H), 1.95 (dd, J=1.5, 17.6, 1H), 1.57 (d, J=5.1 Hz, 3H), 1.52 (m, 1H), 1.47 (m, 1H), 1.43 (s, 9H), 1.09 (m, 2H), 0.86 (t, J=6.7 Hz, 3H), 0.50 (s, 3H).

### EXAMPLE 18

### (4S,5R)-5-((1R,2S)-1-amino-2-methoxy-2-methylpentyl)-4-((1Z)-1-propenyl-2-pyrrolidinone

The solution of Example 17 was cooled to −20° C., treated sequentially with HCl gas (100 g) over 1 hour, stirred for an additional 10 minutes, treated with diatomaceous earth (Celite®, 50 g), stirred for 5 minutes, concentrated to 500 mL, treated with water (200 mL) and toluene (200 mL), and filtered. The organic layer was isolated and extracted with water (100 mL). The extracts were combined and washed with toluene (200 mL) to provide a solution of the desired product, which was used directly in the next step.

### EXAMPLE 19

### N-((1R,2S)-2-methoxy-2-methyl-1-((2R,3S)-5-oxo-3-((1Z)-1-propenyl)pyrrolidinyl)pentyl)acetamide

The combined aqueous extracts from Example 18 were treated with 50% (w/w) aqueous NaOH (17 mL) to pH 14 and acetic anhydride (16.62 g and 1.48 g), with additional 50% NaOH added to maintain the pH of the solution at 12, stirred for 5 minutes, and treated with sodium chloride (7 g). The organic layer was isolated, and the water layer was extracted with isopropyl acetate (2×200 mL). The organics were combined and concentrated with sequential, intermittent isopropyl acetate (3×200 mL) and THF (250 mL) treatment to provide the desired product, which was used directly in the next step. ¹H NMR (CDCl₃) δ 6.44 (br s, 1H), 5.50 (m, 1H), 5.39 (ddq, J=9.2, 10.7, 1.5 Hz, 1H), 4.26 (dd, J=8.1, 10.3 Hz, 1H), 3.51 (dd, J=6.6, 8.1 Hz, 1H), 3.30 (ddd, J=6.9, 9.1, 16.1 Hz, 1H), 3.18 (s, 3H), 2.45 (dd, J=10.2, 16.9 Hz, 1H), 1.99 (dd, J=8.9, 16.9 Hz, 1H), 1.95 (s, 3H, 1.62 (dd, J=1.5, 6.7 Hz, 3H), 1.44 (m, 2H), 1.43 (br s, 1H), 1.25 (m, 2H), 1.11 (s, 3H), 0.89 (t, J=7.0 Hz, 3H).

### EXAMPLE 20

### tert-butyl (2R,3S)-2-((1R,2S)-1-(acetylamino)-2-methoxy-2-methylpentyl)-5-oxo-3-((1Z)-1-propenyl)-1-pyrrolidinecarboxylate

A solution of the concentrate from Example 19 in THF (250 mL) was treated sequentially with triethylamine (23.5 mL), N,N-dimethylaminopyridine (0.51 g), and di-tert-butyldicarbonate (27.43 g) in THF (20 mL), stirred for 3.5 hours, cooled to −10° C., and treated with 0.5M HCl (310 mL) over 25 minutes. The water layer (pH 6.44) was isolated and extracted with isopropyl acetate (2×200 mL). The organics were combined, washed with 23% (w/w) aqueous NaCl, concentrated to 100 mL, treated with isopropyl acetate (300 mL), concentrated to 200 mL, treated with heptane (200 mL), and concentrated to 50-60 mL to provide a slurry. The slurry was stirred for 20 minutes at ambient temperature and filtered with cold (0° C.) 1:1 heptane/isopropyl acetate (2×100 mL) rinsing. The filtrant was dried under vacuum at 50° C. to provide 26.80 g (82%, 4 steps) of the desired product. ¹H NMR (CDCl₃) δ 5.94 (br d, J=9.2 Hz, 1H), 5.45 (m, 2H), 4.55 (dd, J=2.5, 9.5 Hz, 1H), 4.03 (dd, J=0.7, 2.6 Hz, 1H), 3.81 (dt, J=0.7, 9.2 Hz, 1H), 3.24 (s, 3H), 2.78 (dd, J=9.5, 18.0 Hz, 1H), 2.06 (dd, J=1.5, 18 Hz, 1H), 2.00 (s, 3H), 1.79 (dd, J=8.8, 12.8 Hz, 1H), 1.71 (m, 2H), 1.67 (d, J=5.1 Hz, 1H), 1.60 (m, 2H), 1.58 (s, 9H), 1.27 (dd, J=7.0, 8.8 Hz, 1H), 1.16 (s, 3H), 0.95 (t, J=6.9 Hz, 3H).

### EXAMPLE 21

### 2:1 anomeric tert-butyl (2R,3S)-2-((1R,2S)-1-(acetylamino)-2-methoxy-2-methylpentyl)-5-hydroxy-3-((1Z)-1-propenyl)-1-pyrrolidinecarboxylate

A solution of Example 20 (50.0 g) in THF (500 mL) at −13.5° C. with overhead stirring was treated with 1M lithium triethylborohydride (Superhydride®, 157 mL) over 30 minutes, stirred for an additional 15 minutes, treated with methanol (75 mL) over 5 minutes, stirred for an additional 5 minutes at −5° C., warmed to 17° C., stirred for 30 minutes, concentrated to 200 mL, treated with THF (500 mL), concentrated to 100 mL, and added to 5% (w/w) aqueous KH₂PO₄. The organic layer was isolated and washed with 12% (w/w) aqueous NaCl (750 mL) to provide a solution of 48.77 g (97%) of desired product, which was used directly in the next step.

### EXAMPLE 22

### tert-butyl (2R,3S)-2-((1R,2S)-1-(acetylamino)-2-methoxy-2-methylpentyl)-5-methoxy-3-((1Z)-1-propenyl)-1-pyrrolidinecarboxylate

The solution of Example 21 was concentrated twice to 100 mL with a methanol treatment (500 mL) between the concentrations then concentrated to 176.1 g after a final methanol (500 mL) treatment. The concentrate was treated with methanol (90 mL), trimethylorthoformate (50 mL), and camphor sulfonic acid (1.41 g), stirred for 1 hour, treated with 5% (w/w) aqueous KHCO₃, and extracted with isopropyl acetate (1L). The extract was washed with 23% (w/w) aqueous NaCl, concentrated twice to 150 mL with an intermittent acetonitrile treatment (500 mL), concentrated to 150.57 g after a acetonitrile (500 mL) treatment, and treated with acetonitrile (370 mL) to provide 435.5 g of a 11.2% (w/w) solution of the desired product (97.5%), which was used directly in the next step. ¹H NMR (CDCl₃) δ 5.98 (br d, J=9.9 Hz, 1H), 5.43 (m, 1H), 5.31 (m, 1H), 5.17 (d, J=5.5 Hz, 0.5H), 5.01 (d, J=5.1 Hz, 0.5H), 4.72 (dd, J=2.2, 9.9 Hz, 1H), 3.93 (dd, J=3.0, 8.1 Hz, 0.5H), 3.82 (dd, J=2.9, 7.7 Hz, 0.5H), 3.33 (s, 1.5H), 3.30 (s, 1.5H), 3.06 (s, 3H), 2.05 (s, 1.5H), 2.03 (s, 1.5H), 1.97 (m, 2H), 1.88 (m, 1H), 1.79 (m, 2H), 1.63 (dd, J=1.5, 6.6 Hz, 3H), 1.56 (s, 4.5H), 1.52 (m, 1H), 1.49 (s, 4.5H), 1.23 (m, 1H), 1.12 (s, 3H), 0.95 (t, J=6.9 Hz, 3H).

### EXAMPLE 23

### tert-butyl (2R,3S,5R)-2-((1R,2S)-1-(acetylamino)-2-methoxy-2-methylpentyl)-5-cyano-3-((1Z)-1-propenyl)-1-pyrrolidinecarboxylate

A solution 13% (w/w) of Example 22 in acetonitrile (153.66 g) was treated with acetonitrile (23.83 g), cooled to −20° C., treated with trimethylsilylcyanide (15.19 g), cooled to −40° C., treated with trifluoromethanesulfonic acid (10.95 g) over 30 minutes, stirred an additional 3 hours, and added to a mixture of 10% (w/w) aqueous K₂CO₃ (400 mL) containing 7.76 g NaOH and isopropyl acetate (400 mL). The extract was separated, washed sequentially with 23% (w/w) aqueous NaCl and 23% (w/w) aqueous NaCl containing KH₂PO₄ (2.82 g) and Na₂HPO₄.7H₂O (7.12 g), concentrated to 50 mL with intermittent isopropyl alcohol treatment (3×400 mL), and treated with isopropyl alcohol to provide 95.74 g of a 17.2% (w/w) solution of the desired product (86.9%), which was used directly in the next step. ¹H NMR (CDCl₃) δ 5.93 (br d, J=9.2 Hz, 1H), 5.72 (m, 1H), 5.50 (m, 1H), 4.55 (dd, J=1.5, 9.2 Hz, 1H), 4.42 (ddd, J=1.1, 9.5, 27.2 Hz, 1H), 3.89 (d, J=1.4Hz, 1H), 3.75 (m, 1H), 3.20 (s, 3H), 2.50 (ddd, J=7.7, 9.1, 17.2 Hz, 1H), 2.00 (s, 3H), 1.95 (d, J=13.2 Hz, 1H), 1.72 (m, 1H), 1.69 (m, 1H), 1.66 (dd, J=1.8 7.0 Hz, 3H), 1.55 (s, 9H), 1.30 (m, 1H), 1.21 (m, 1H), 1.13 (s, 3H), 0.95 (t, J=6.6 Hz, 3H).

### EXAMPLE 24

### isopropyl (2R,5R)-5-((1R,2S)-1-(acetylamino)-2-methoxy-2-methylpentyl)-4-((1Z)-1-propenyl)-2-pyrrolidinecarboxylate, para-toluenesulfonic acid salt

A solution of 20% (w/w) of Example 23 in isopropyl alcohol (231.43 g) was treated with isopropyl alcohol (44.43 g), cooled to −30° C., treated with HCl gas (100-200 g) over 23 minutes, warmed to ambient temperature over 1 hour, treated with isopropyl alcohol (235 g), refluxed for 8 hours, cooled to ambient temperature, and concentrated twice to one-half of its original volume with one intermittent isopropyl alcohol treatment (245 mL). The concentrate was added to 15% (w/w) aqueous KHCO₃ (689 g) with isopropyl acetate (248 g) and water (50 g) rinsing and stirred for 15 minutes. The organic layer was isolated and washed with 15% (w/w) aqueous KHCO₃ (376 g). The aqueous washings were combined and extracted with isopropyl acetate (160.9 g), and the combined organic extracts were washed with 20% (w/w) aqueous NaCl (300 g) and concentrated twice to one-quarter of their original volume with an intermittent isopropyl acetate treatment (374 g). This concentrate was filtered with isopropyl acetate (427 g) rinsing. The filtrate was treated with isopropyl acetate (843.56 g), warrned to 50-60° C., and treated with a solution ofpara-toluenesulfonic acid monohydrate (24.07 g) in isopropyl acetate (256.7 g) at 50-60° C., stirred for 50 minutes, cooled to ambient tremperature at 5° C./hour, and filtered with isopropyl acetate (2×200 g) rinsing. The filtrant was dried under vacuum at 40° C. to provide 53.78 g (94.7%) of the desired product. ¹H NMR (D₂O) δ 7.73 (d, J=8.5 Hz, 2H), 7.41 (d, J=8.5 Hz, 2H), 5.69 (ddq, J=7.0, 11.0, 1.1 Hz, 1H), 5.33 (ddq, J=9.2, 11.0, 1.9 Hz, 1H), 5.16 (septet, J=6.2 Hz, 1H), 4.49 (dd, J=7.7, 9.5 Hz, 1H), 4.43 (d, J=10.3 Hz, 1H), 3.75 (t, J=10.0 Hz, 1H), 3.28 (s, 3H), 3.25 (m, 1H), 2.66 (dt, J=13.6, 7.7 Hz, 1H), 2.44 (s, 3H), 2.00 (s, 3H), 1.82 (dt, J=13.6, 10.3 Hz, 1H), 1.68 (m, 1H), 1.61 (dd, J=1.9, 7.0 Hz, 3H), 1.41 (m, 1H), 1.34 (d, J=6.3 Hz, 3H), 1.33 (d, J=6.3 Hz, 3H), 1.30 (m, 2H), 1.27 (s, 3H), 0.88 (t, J=7.0 Hz, 3H).

It will be evident to one skilled in the art that the processes and compounds of this invention are not limited to the forgoing examples, and that these processes and compounds can generally be embodied in other specific forms without departing from the essential attributes thereof, namely by substitution of the appropriate reactants and agents. Thus, it is desired that the examples be considered as illustrative and not restrictive, reference being made to the claims and not the foregoing examples, and that all changes which come within the meaning and range of equivalency of the claims be embraced therein.

What is claimed is:

1. A process for making a compound having structural formula (5) 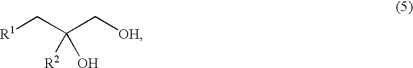
in which R¹ is alkyl, cycloalkyl, cycloalkylalkyl, or arylalkyl; and
R² is alkyl, cycloalkyl, cycloalkylalkyl, or arylalkyl, comprising, in toto, the steps of:
(c) reacting a compound having structural formula (3) 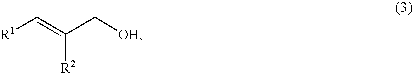
and a titanium iso-propoxidedialkyl tartrate complex;
(d) reacting the product of step (c) and tert-butyl hydroperoxide to provide a compound having structural formula (4), 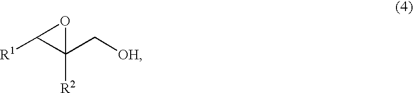
(e) reacting the product of step (d) and a second reducing agent to provide the compound having structural formula (5);
and
(f) optionally isolating the compound having structural formula (5).

2. The process of claim 1 in which steps (c) and (d) are conducted sequentially.

3. The process of claim 1 in which steps (d) and (e) are conducted continuously.

4. The process of claim 1 in which the compound having the structural formula (4) has the stereochemistry illustrated by a compound having structural formula (4-a) 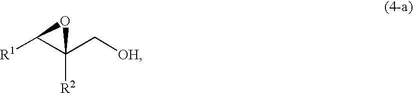
and the compound having the structural formula (5) has the stereochemistry illustrated by a compound having structural formula (5-a)

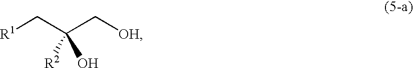

5. The process of claim 1 in which the compound having the structural formula (3) is substantially pure (2E)-2-methyl-2-penten-1-ol;
the compound having the structural formula (4) is substantially pure, enantiomerically enriched 3,4-anhydro-1,2-dideoxy-4-methyl-D-threo-pentitol;
and the compound having the structural formula (5) is substantially pure, enantiomerically enriched (2S)-2-methyl-1,2-pentanediol.

6. The process of claim 1 further comprising making the compound having formula (3) comprising, in toto, the steps of:
(a) reacting a compound having structural formula (1) 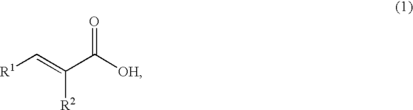
a first acid, a first esterifying agent, and, optionally, a trialkylorthoformate to provide a compound having structural formula (2) 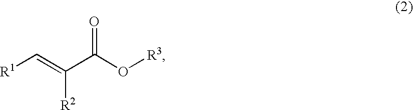
in which R³ is C₁-C₄ alkyl;
and
(b) reacting the product of step (a) and a first reducing agent to provide the compound having structural formula (3).

7. A process for making a compound having structural formula (12) 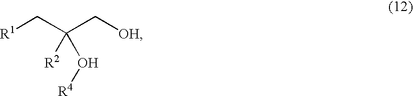
in which R¹ is alkyl, cycloalkyl, cycloalkylalkyl, or arylalkyl;
R² is alkyl, cycloalkyl, cycloalkylalkyl, or arylalkyl; and
R⁴ is alkyl, cycloalkyalkyl, or aryl-(C₂-C₄-alkyl), comprising, in toto, the steps of:
(g) reacting a compound having structural formula (5) 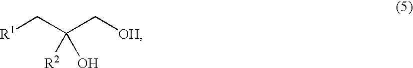
a first base, and a selectively removable hydroxy protecting group precursor to provide a compound having structural formula (6) 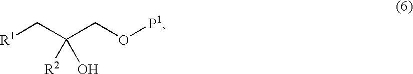
in which P¹ is a selectively removable hydroxy protecting group;
(h) reacting the product of step (g), an alkylating agent, and the first base to provide a compound having structural formula (7) 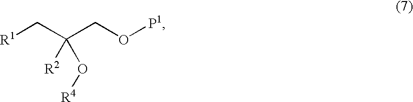
(i) reacting the product of step (h) and a hydroxy protecting group removal agent to provide the compound having structural formula (12);
and
(j) optionally isolating the compound having structural formula (12).

8. The process of claim 7 in which steps (g) and (h) are conducted sequentially.

9. The process of claim 7 in which steps (h) and (i) are conducted continuously.

10. The process of claim 7 in which the compound having the structural formula (5) has the stereochemistry illustrated by a compound having structural formula (5-a) 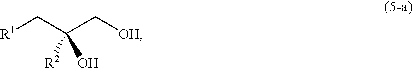
the compound having the structural formula (6) has the stereochemistry illustrated by a compound having structural formula (6-a) 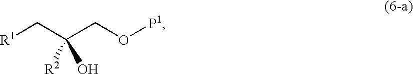
the compound having the structural formula (7) has the stereochemistry illustrated by a compound having structural formula (7-a) 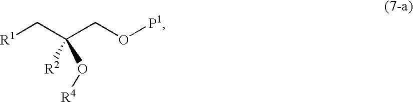
and the compound having the structural formula (12) has the stereochemistry illustrated by a compound having structural formula (12-a)

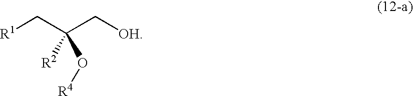

11. The process of claim 7 in which the compound having the structural formula (5) is substantially pure, enantiomerically enriched (2S)-2-methyl-1,2-pentanediol;
the compound having the structural formula (6) is substantially pure, enantiomerically enriched (2S)-1-(benzyloxy)-2-methyl-2-pentanol;
the compound having the structural formula (7) is substantially pure, enantiomerically enriched ((((2S)-2-methoxy-2-methylpentyl)oxy)methyl)benzene;
and the compound having the structural formula (12) is substantially pure, enantiomerically enriched (2S)-2-methoxy-2-methyl-1-pentanol.

12. A process for making an enantiomerically enriched compound having structural formula (12-b) 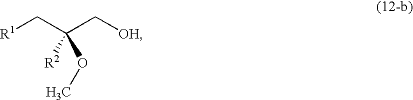
having the stereochemistry illustrated therein, in which
R¹ is alkyl, cycloalkyl, cycloalkylalkyl, or arylalkyl; and
R² is alkyl, cycloalkyl, cycloalkylalkyl, or arylalkyl, comprising, in toto, the steps of:
(m) reacting a compound having structural formula (10) 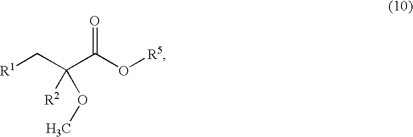
in which R⁵ is alkyl, C₂-C₄-haloalkyl, cycloalkylalkyl, or arylalkyl, and an esterase to provide either an enantiomerically enriched compound having structural formula (11-a) 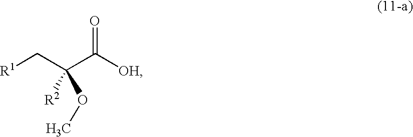
or an enantiomerically enriched compound having structural formula (11-b) 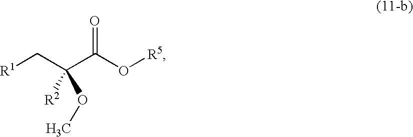
(n) reacting the compound having structural formula (11-a) or the compound of (11-b) and a third reducing agent to provide the compound having structural formula (12-b);
and
(o) optionally isolating the compound having structural formula (12-b).

13. The process of claim 12 in which steps (m) and (n) are conducted continuously.

14. The process of claim 12 in which the compound having the structural formula (10) is substantially pure methyl 2-methoxy-2-methylpentanoate;
the compound having the structural formula (11) is substantially pure, enantiomerically enriched (2S)-2-methoxy-2-methylpentanoic acid;
and the compound having the structural formula (12-b) is substantially pure, enantiomerically enriched (2S)-2-methoxy-2-methyl-1-pentanol.

15. The process of claim 12 further comprising making the compound having formula structural (10) comprising, in toto, the steps of:
(k) reacting a compound having structural formula (8) 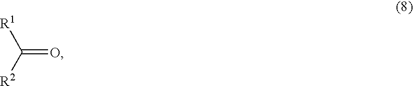
bromoform, potassium hydroxide, and methanol to provide a compound having structural formula (9) 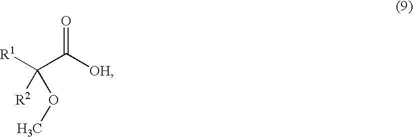
and
(l) reacting the product of step (k), a second esterifying agent and, optionally, an esterification promotion agent to provide the compound having structural formula (10).

16. A process for making a compound having structural formula (15) 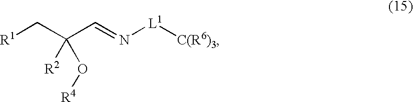
in which R¹ is alkyl, cycloalkyl, cycloalkylalkyl, or arylalkyl;
R² is alkyl, cycloalkyl, cycloalkylalkyl, or arylalkyl;
R⁴ is alkyl, cycloalkyalkyl, or aryl-(C₂-C₄-alkyl);
L¹ is N(R⁷), O, S, S(O), or SO₂;
R⁶ is aryl, faranyl, or, thienyl in which the aryl, the furanyl, and the thienyl are unsubstituted or substituted with one, two, or three substituents independently selected from the group consisting of alkyl, alkoxy, halo, and nitro; and
R⁷ is hydrogen, alkyl, aryl, orpara-toluenesulfonyl. comprising, in toto, the steps of:
(p) reacting a compound having structural formula (12) 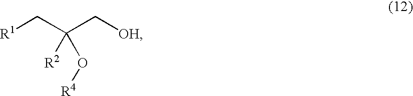
an oxidant, a second base, and, optionally, a first additive, in which the oxidant, the second base, and the first additive are substantially soluble in the solvent or solvents in which this step is conducted, to provide a compound having structural formula (13) 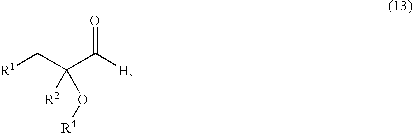
and
(q) reacting the product of step (p), a compound having structural formula (14) 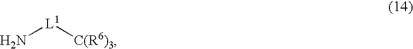
a drying agent, and, optionally, a first acid catalyst to provide the compound having structural formula (15).

17. The process of claim 16 in which steps (p) and (q) are conducted in situ.

18. The process of claim 16 in which the compound having the structural formula (12) has the stereochemistry illustrated by the compound having structural formula (12-a) 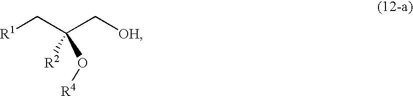
the compound having the structural formula (13) has the stereochemistry illustrated by a compound having structural formula (13-a) 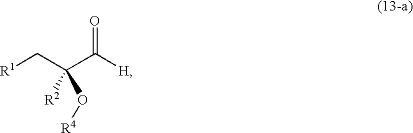
and the compound having the structural formula (15) has the stereochemistry illustrated by a compound having structural formula (15-a)

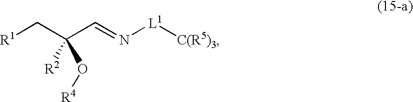

19. The process of claim 16 in which the compound having structural formula (12) is substantially pure, enantiomerically enriched (2S)-2-methoxy-2-methyl-1 -pentanol;
the compound having structural formula (13) is substantially pure, enantiomerically enriched (2S)-2-methoxy-2-methylpentanal;
and the compound having structural formula (15) is substantially pure, enantiomerically enriched (1E,2S)-2-methoxy-2-methylpentanal S-tritylthioxime.

20. A process for making substantially pure (±)-tert-butyl-4-hydroxy-2-oxo-pyrrolidinecarboxylate comprising, in toto, the steps of:
(r) reacting (±)-4-amino-3-hydroxy-n-butyric acid, a silating agent, and a third base to provide (±)-4-trimethylsilyloxy-2-oxo-pyrrolidine;
(s) reacting the (±)-4-trimethylsilyloxy-2-oxo-pyrrolidine and a tert-butylcarbonyloxy-introducing agent, the third base, and 4-dimethylaminopyridine to provide, (±)-tert-butyl-4-trimethylsilyloxy-2-oxo-pyrrolidinecarboxylate;
(t) reacting the (±)-tert-butyl-4-trimethylsilyloxy-2-oxo-pyrrolidinecarboxylate and a desilylating agent to provide (±)-tert-butyl-4-hydroxy-2-oxo-pyrrolidinecarboxylate;
and
(u) isolating the substantially pure (±)-tert-butyl-4-hydroxy-2-oxo-pyrrolidine-carboxylate.

21. The process of claim 20 in which steps (r), (s), (t), and (u) are conducted sequentially.

22. A process for making a compound having structural formula (19-a) 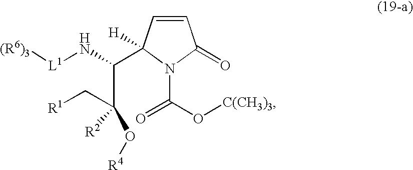
having the absolute stereochemistry illustrated therein,
in which the substituents at the carbons labeled “1” and “2” are erythro relative to each other and “anti-” relative to the O—R⁴ group at the carbon labeled “3,” and in which
R¹ is alkyl, cycloalkyl, cycloalkylalkyl, or arylalkyl;
R² is alkyl, cycloalkyl, cycloalkylalkyl, or arylalkyl;
R⁴ is alkyl, cycloalkyalkyl, or aryl-(C₂-C₄-alkyl);
L¹ is N(R⁷), O, S, S(O), or SO₂;
R⁶ is aryl, furanyl, or thienyl, in which the aryl, the furanyl, and the thienyl are unsubstituted or substituted with one, two, or three substituents independently selected from the group consisting of alkyl, alkoxy, halo, and nitro; and
R⁷ is hydrogen, alkyl, aryl, orpara-toluenesulfonyl, comprising, in toto, the steps of:
(v) reacting (±)-tert-butyl-4-hydroxy-2-oxo-pyrrolidinecarboxylate, a fourth base, and a compound having formula (16)
X¹—SO₂-R⁹ (16),
in which R⁹ is alkyl, haloalkyl, and aryl, in which the aryl is unsubstituted or substituted with one, two, or three substituents independently selected from the group consisting of alkyl, alkoxy, halo, and nitro; and
X¹ is Br, Cl, or OSO₂-R⁹, to provide tert-butyl 2-oxo-3-pyrroline carboxylate;
(w) reacting the tert-butyl 2-oxo-3-pyrroline carboxylate, a fifth base, and a compound having formula (17)
(R⁸)₃SiOSO₂CF₃ (17),
in which each R⁸ is independently selected from the group consisting of alkyl and unsubstituted aryl,
to provide a compound having structural formula (18) 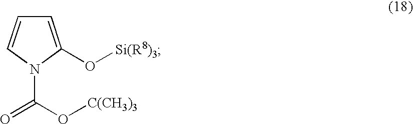
(x) reacting the product of step (w), a second acid, and a substantially enantiomerically enriched compound having structural formula (15-a) 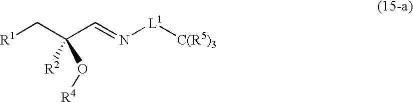
to provide a mixture comprising the compound having structural formula (19-a) in equilibrium with a compound having structural formula (19-b) 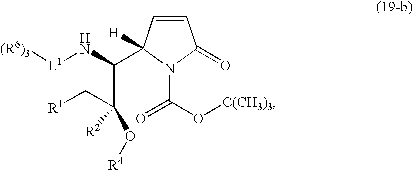
in which the substituents at the carbons labeled “1” and “2” are also erythro relative to each other but are “syn-” relative to the O—R⁴ group at the carbon labeled “3,” and
in which the (19-a)/(19-b) ratio in the mixture is about 4.3:1 to about 10:1;
(y) allowing the mixture of the compound having structural formula (19-a) and the compound having structural formula (19-b) to further equilibrate until the (19-a)/(19-b) ratio in the mixture is greater than 10:1;
and
(z) isolating the compound having structural formula (19-a).

23. The process of claim 22 in which steps (v) and (w) are conducted in situ.

24. The process of claim 22 in which steps (x) and (y) are conducted sequentially.

25. The process of claim 22 in which the compound having structural formula (15-a) is substantially pure, substantially enantiomerically enriched (1E,2S)-2-methoxy-2-methylpentanal S-tritylthioxime;
the compound having structural formula (17) is substantially pure tert-butyldimethylsilyl trifluoromethanesulfonate;
the compound having structural formula (18) is substantially pure tert-butyl 2-((tert-butyl(dimethyl)silyl)oxy)-1H-pyrrole-1-carboxylate;
the compound having structural formula (19-a) is substantially pure, diastereomerically enriched tert-butyl (2R)-2-((1R,2S)-2-methoxy-2-methyl-1-((tritylsulfanyl)amino)pentyl)-5-oxo-2,5-dihydro-1-1H-pyrrole-1-carboxylate;
and the compound having structural formula (19-b) is substantially pure, diastereomerically enriched tert-butyl (2S)-2-((1S,2S)-2-methoxy-2-methyl-1-((tritylsulfanyl)amino)pentyl)-5-oxo-2,5-dihydro-1-1H-pyrrole-1-carboxylate.

26. A process for making a compound having structural formula (24) 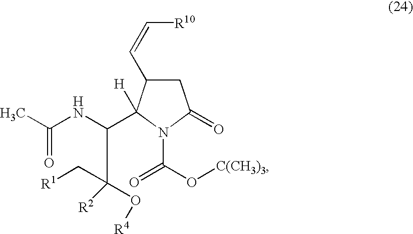
in which the carbon-carbon double bond is substantially in the Z configuration,
and in which R¹ is alkyl, cycloalkyl, cycloalkylalkyl, or arylalkyl;
R² is alkyl, cycloalkyl, cycloalkylalkyl, or arylalkyl;
R⁴ is alkyl, cycloalkyalkyl, or aryl-(C₂-C₄-alkyl); and
R¹⁰ is methyl, ethyl, iso-propyl, or vinyl, comprising, in toto, the steps of:
(aa) reacting a compound having structural formula (19) 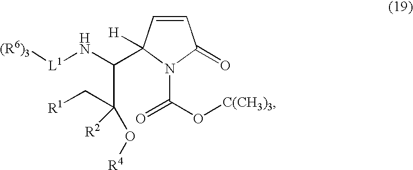
a conjugate addition agent said conjugate addition agent comprising a mixture of compounds having structural formula (20-Z)
and structural formula (20-E) 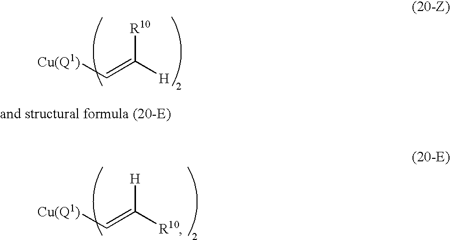
in which the (20-Z)/(20-E) ratio in the mixture is about 97:3, and
in which Q¹ is Li, Mg—Cl, Mg—Br, or Mg—I, and, optionally, a second additive at a temperature between about −50° C. and about −30° C. about to provide a compound having structural formula (21) 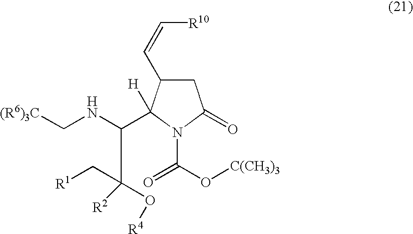
in which the carbon-carbon double bond is substantially in the Z configuration;
(bb) reacting the product of step (aa) and a third acid to provide a compound having the structural formula (22) 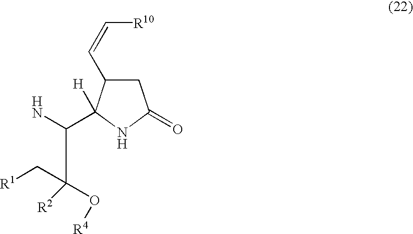
or a salt thereof;
(cc) reacting the product of step (bb), an acetylating agent, and a sixth base to provide a compound having the structural formula (23) 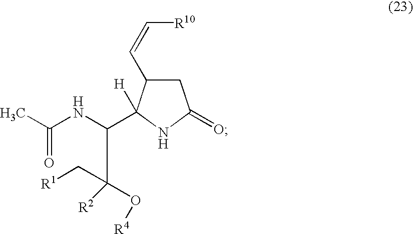
(dd) reacting the product of step (cc), a tert-butylcarbonyloxy-introducing agent, a seventh base, and 4-dimethylaminopyridine to provide the compound having formula (24);
and
(ee) optionally isolating the product of step (dd).

27. The process of claim 26 in which steps (aa)-(dd) are conducted in situ.

28. The process of claim 26 in which the compound having the structural formula (19) has the absolute stereochemistry illustrated by a compound having structural formula (19-a) 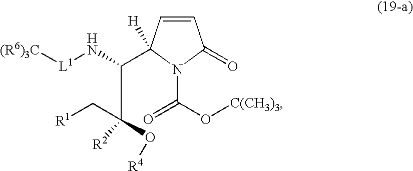
the compound having the structural formula (21) has the absolute stereochemistry illustrated by a compound having structural formula (21-a) 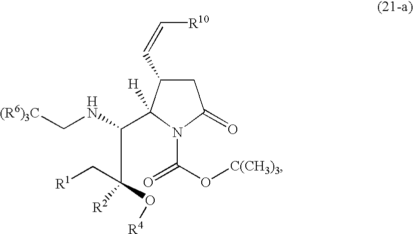
the compound having the structural formula (22) has the absolute stereochemistry illustrated by a compound having structural formula (22-a) 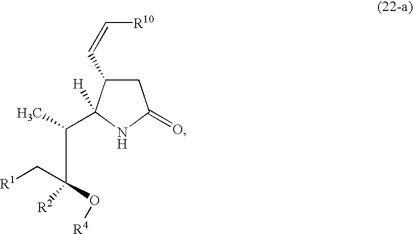
the compound having the structural formula (23) has the absolute stereochemistry illustrated by a compound having structural formula (23-a) 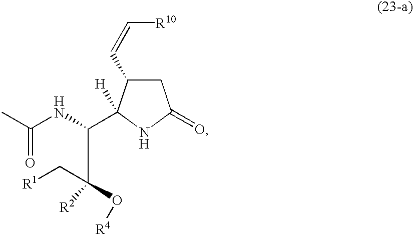
and the compound having the structural formula (24) has the absolute stereochemistry illustrated by a compound having structural formula (24-a)

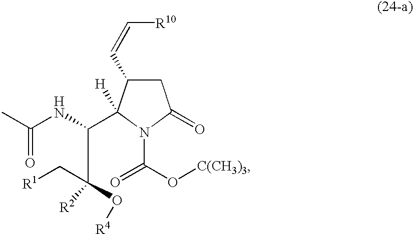

29. The process of claim 26 in which the compound having structural formula (19) is substantially pure, diastereomerically enriched tert-butyl (2R)-2-((1R,2S)-2-methoxy-2-methyl-1-((tritylsulfanyl)amino)pentyl)-5-oxo-2, 5-dihydro-1-1-1H-pyrrole-1-carboxylate;
the compound having structural formula (21) is substantially pure, substantially diastereomerically enriched tert-butyl (2R,3S)-2-((1R,2S)-2-methoxy-2-methyl-1-((tritylsulfanyl)amino)pentyl)-5-oxo-3-((1Z)-1-propenyl)-1-pyrrolidinecarboxylate;
the compound having structural formula (22) is substantially pure, substantially diastereomerically enriched (4S,5R)-5-((1R,2S)-1-amino-2-methoxy-2-methylpentyl)-4-((1Z)-1-propenyl)-2-pyrrolidinone;
the compound having structural formula (23) is substantially pure, substantially diastereomerically enriched N-((1R,2S)-2-methoxy-2-methyl-1-((2R,3S)-5-oxo-3-((1Z)-1-propenyl)pyrrolidinyl)pentyl)acetamide;
and the compound having structural formula (24) is substantially pure, substantially diastereomerically enriched tert-butyl (2R,3S)-2-((1R,2S)-1-(acetylamino)-2-methoxy-2-methylpentyl)-5-oxo-3-((1Z)-1-propenyl)-1-pyrrolidinecarboxylate.

30. A process for making a compound having structural formula (28) 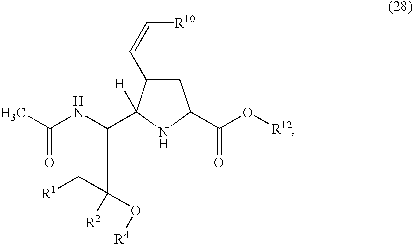
or a therapeutically acceptable salt thereof,
in which R¹ is alkyl, cycloalkyl, cycloalkylalkyl, or arylalkyl;
R² is alkyl, cycloalkyl, cycloalkylalkyl, or arylalkyl;
R⁴ is alkyl, cycloalkyalkyl, or aryl-(C₂-C₄-alkyl);
R¹⁰ is methyl, ethyl, iso-propyl, or vinyl; and
R¹² is hydrogen or alkyl, comprising, in toto, the steps of:
(ff) reacting a compound having structural formula (24) 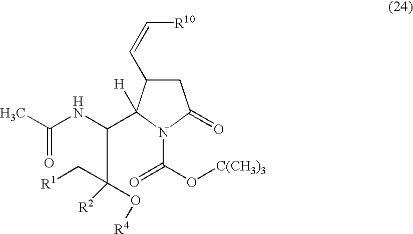
and a fourth reducing agent to provide a compound having structural formula (25) 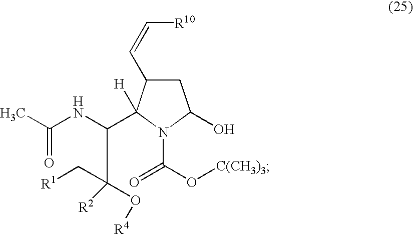
(gg) optionally reacting the product of step (ff), a C₁-C₅ alcohol, a trialkylorthoformate, and a second acid catalyst to provide a compound having structural formula (26) 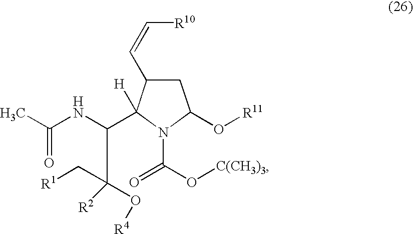
in which R¹ is methyl, ethyl, propyl, or iso-propyl;
(hh) reacting the product of step (ff) or step (gg), a cyanide-donating agent, and a fourth acid to provide a compound having structural formula (27) 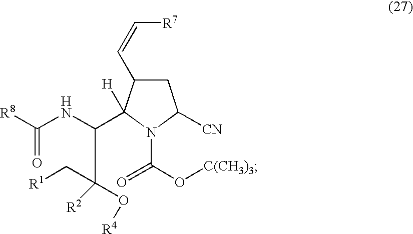
(ii) reacting the product of step (hh), a C₁-C₅ alcohol, and a fifth acid to provide the compound having structural formula (28), or a therapeutically acceptable salt thereof;
and
(jj) isolating the product of step (ii).

31. The process of claim 30 in which steps (ff), (gg), (hh), and (ii) are conducted in situ.

32. The process of claim 30 in which the compound having the structural formula (24) has the absolute stereochemistry illustrated by a compound having structural formula (24-a) 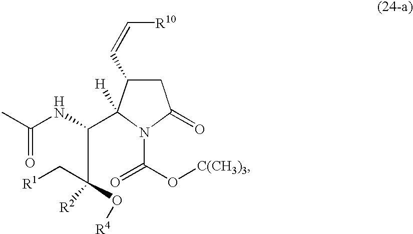
the compound having the structural formula (25) has the absolute stereochemistry illustrated by a compound having structural formula (25-a) 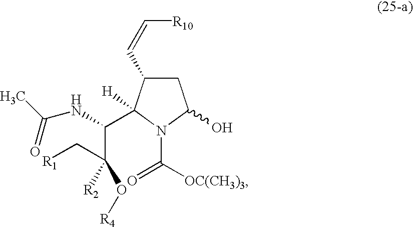
the compound having the structural formula (26) has the absolute stereochemistry illustrated by a compound having structural formula (26-a) 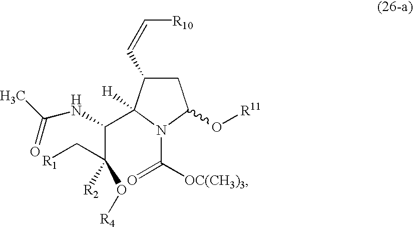
the compound having the structural formula (27) has the absolute stereochemistry illustrated by a compound having structural formula (27-a) 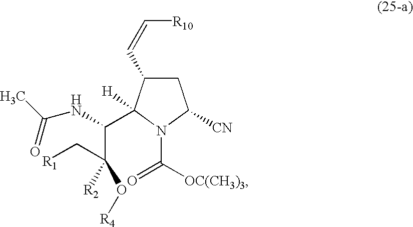
and the compound having the structural formula (28) has the absolute stereochemistry illustrated by a compound having structural formula (28-a)

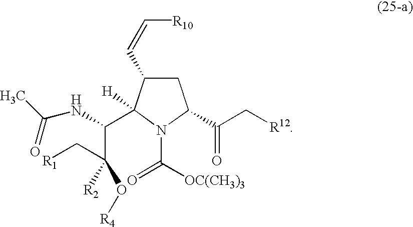

33. The process of claim 30 in which the compound having structural formula (24) is substantially pure, diastereomerically enriched tert-butyl (2R,3S)-2-((1R,2S)-1-(acetylamino)-2-methoxy-2-methylpentyl)-5-oxo-3-((1Z)-1-propenyl)-1-pyrrolidinecarboxylate;
the compound having structural formula (25) is substantially pure, diastereomerically enriched 2:1 anomeric tert-butyl (2R,3S)-2-((1R,2S)-1-(acetylamino)-2-methoxy-2-methylpentyl)-5-hydroxy-3-((1Z)-1-propenyl)-1-pyrrolidinecarboxylate;
the compound having structural formula (26) is substantially pure, diastereomerically enriched tert-butyl (2R,3S)-2-((1R,2S)-1-(acetylamino)-2-methoxy-2-methylpentyl)-5-methoxy-3-((1Z)-1-propenyl)-1-pyrrolidinecarboxylate;
the compound having structural formula (27) is substantially pure, diastereomerically enriched tert-butyl (2R,3S,5R)-2-((1R,2S)-1-(acetylamino)-2-methoxy-2-methylpentyl)-5-cyano-3-((1Z)-1-propenyl)-1-pyrrolidinecarboxylate;
and the compound having structural formula (28) is substantially pure, diastereomerically enriched isopropyl (2R,5R)-5-((1R,2S)-1-(acetylamino)-2-methoxy-2-methylpentyl)-4-((1Z)-1-propenyl-2-pyrrolidinecarboxylate, para-toluenesulfonic acid salt.

34. A compound selected from the group consisting of a compound having structural formula (29) 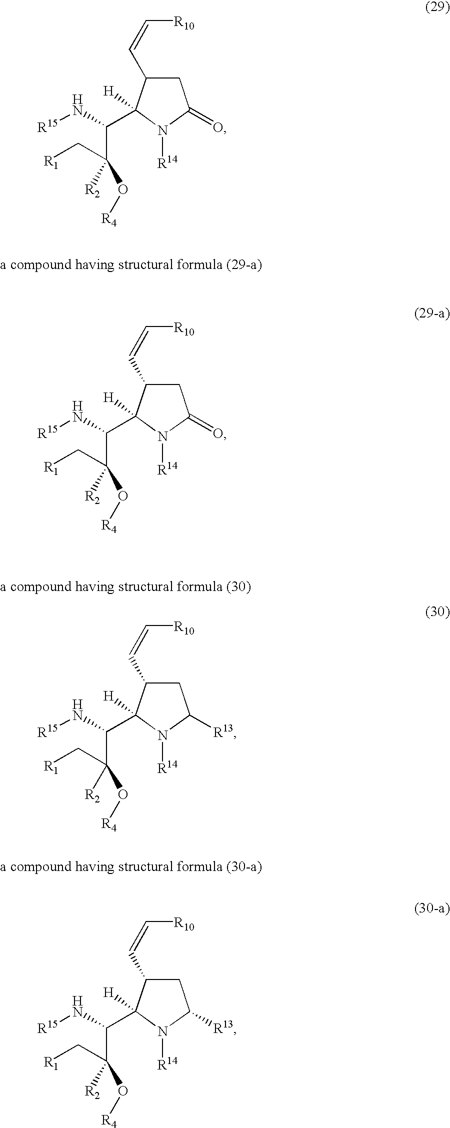
or a salt thereof,
in which the carbon-carbon double bond in the compounds having structural formulas (29), (29-a), (30), and (30-a) is substantially in the Z configuration, and
in which
R¹ is alkyl, cycloalkyl, cycloalkylalkyl, or arylalkyl;
R² is alkyl, cycloalkyl, cycloalkylalkyl, or arylalkyl;
R⁴ is alkyl, cycloalkyalkyl, or aryl-(C₂-C₄-alkyl);
R¹⁰ is methyl, ethyl, iso-propyl, or vinyl;
R¹³ is alkoxy alkoxycarbonyl, and hydroxy;
R¹⁴ is hydrogen or tert-butoxycarbonyl; and
R¹⁵ is hydrogen or acetyl,
with the proviso that R¹³ is alkoxycarbonyl only for the compound having structural formula (30-a).

35. A compound of claim 34 having structural formula (29) in which R¹ is alkyl; R² is alkyl; R⁴ is alkyl; R¹⁴ is hydrogen, and R¹⁵ is hydrogen.

36. A compound of claim 35 which is (4S,5R)-5-((1R,2S)-1-amino-2-methoxy-2-methylpentyl)-4-((1Z)-1-propenyl)-2-pyrrolidinone.

37. A compound of claim 34 having structural formula (29) in which R¹ is alkyl; R² is alkyl; R⁴ is alkyl; R¹⁴ is hydrogen, and R¹⁵ is acetyl.

38. A compound of claim 37 which is N-((1R,2S)-2-methoxy-2-methyl-1-((2R,3S)-5-oxo-3-((1Z)-1-propenyl)pyrrolidinyl)-pentyl)acetamide.

39. A compound of claim 34 having structural formula (29) in which R¹ is alkyl; R² is alkyl; R⁴ is alkyl; R¹⁴ is tert-butoxycarbonyl, and R¹⁵ is acetyl.

40. A compound of claim 39 which is tert-butyl (2R,3S)-2-((1R,2S)-1-(acetylamino)-2-methoxy-2-methylpentyl)-5-oxo-3-((1Z)-1-propenyl)-1-pyrrolidinecarboxylate.

41. A compound of claim 34 having structural formula (30) in which R¹ is alkyl; R² is alkyl; R⁴ is alkyl; R¹³ is alkoxy; R¹⁴ is tert-butoxycarbonyl, and R¹⁵ is acetyl.

42. A compound of claim 41 which is anomeric tert-butyl (2R,3S)-2-((1R,2S)-1-(acetylamino)-2-methoxy-2-methylpentyl)-5-hydroxy-3-((1Z)-1-propenyl)-1-pyrrolidinecarboxylate.

43. A compound of claim 34 having structural formula (30) in which R¹ is alkyl; R² is alkyl; R⁴ is alkyl; R¹³ is hydroxy; R¹⁴ is tert-butoxycarbonyl, and R¹⁵ is acetyl.

44. A compound of claim 43 which is tert-butyl (2R,3S)-2-((1R,2S)-1-(acetylamino)-2-methoxy-2-methylpentyl)-5-methoxy-3-((1Z)-1-propenyl)-1-pyrrolidinecarboxylate.

45. A compound of claim 34 having structural formula (30-a) in which R¹ is alkyl; R² is alkyl; R⁴ is alkyl; R¹³ is alkoxycarbonyl; R¹⁴ is hydrogen, and R¹⁵ is acetyl.

46. A compound of claim 45 which is isopropyl (2R,5R)-5-((1R,2S)-1-(acetylamino)-2-methoxy-2-methylpentyl)-4-((1Z-1-propenyl)-2-pyrrolidinecarboxylate, para-toluenesulfonic acid salt.
